(19) United States
(12) Patent Application Publication
(10) Pub. No.: US 2021/0361578 A1
(43) Pub. Date: Nov. 25, 2021

(54) MEK1/2 INHIBITOR-LOADED MICROPARTICLE FORMULATION

(71) Applicants: University of Iowa Research Foundation, Iowa City, IA (US); The United States Government, as Represented by the Department of Veterans Affairs, Washington, DC (US)

(72) Inventors: Aliasger K. Salem, Coralville, IA (US); Robert Felder, Iowa City, IA (US); Youssef Wahib Naguib Ibrahim, Iowa City, IA (US)

(21) Appl. No.: 17/221,532

(22) Filed: Apr. 2, 2021

Related U.S. Application Data
(60) Provisional application No. 63/004,975, filed on Apr. 3, 2020.

(51) Int. Cl. A61K 9/16; A61K 9/00; A61P 9/04; A61K 31/352; A61K 31/277; A61K 31/44; A61K 31/4523; A61K 31/4184; A61K 31/18; A61K 31/519; A61K 31/437; A61K 31/4412

(52) U.S. Cl. CPC A61K 9/1647; A61K 9/0019; A61P 9/04; A61K 31/352; A61K 31/277; A61K 31/4412; A61K 31/4523; A61K 31/4184; A61K 31/18; A61K 31/519; A61K 31/437; A61K 31/44

(57) ABSTRACT

A composition comprising microparticles or liposomes comprising one or more MEK1/2 inhibitors, and methods of using the composition, are provided.

### CROSS-REFERENCE TO :RELATED APPLICATIONS

This application claims the benefit of the filing date of U.S. application No. 63/004,975, filed on Apr. 3, 2020, the disclosure of which is incorporated by reference herein.

### STATEMENT OF GOVERNMENT RIGHTS

The invention was made with government support under HL136149 and OD019941 awarded by the National Institutes of Health. The government has certain rights in the invention.

### BACKGROUND

The mitogen-activated protein kinase (MAPK) cascade is a ubiquitous evolutionarily conserved serine/threonine protein kinase pathway essential for multiple cellular processes, including cell survival, proliferation, differentiation, development, apoptosis, metabolism, migration, and senescence (Chang et al., 2001; Pearson et al., 2001; Cseh et at, 2014). This pathway transmits upstream signals from cell membrane receptors to the nucleus via a series of sequential phosphorylation processes (Wit et al., 2015). Following external stimuli-related ligand binding to G-protein coupled receptors in the cell membrane, the signal transmission is initiated by the activation of the GDP/GIP binding protein Ras, which in turn activates (phosphorylates) Raf (also known as MAPK kinase kinase, MAPKKK, ERK kinase, or ERKK) (Chang et al., 2001; Blankat et al., 2000; Neuzilliet et al., 2014). Consequently, activation of MAPKKK activates MEK. (also known as MAPKK or MAPK-ERK kinase), which finally activates one of four different effector protein kinases, namely ERK1/2 (extracellular signal-regulated kinase, also called p42/44), c-JNK (c-jun N-terminal kinase), p38 MAPK, and ERK5. The most widely studied MAPK pathway is the Ras-Raf-MEK-ERK. Mutations in Ras (including the three highly conserved HRas, KRas, and NRas) predispose an individual to many types of cancer (Neuzillet et al., 2014; Li et al., 2016; Costigan et al., 2019; Li S et at., 2018; Savoia et al., 2019; Wang et al, 2013). The MEK-ERK pathway is also activated downstream of other tyrosine kinase receptors highly involved in cancer, such as epithelial growth factor receptor (EGFR) (Baru et al., 2009). While targeting Ras mutations with small molecules may seem far from reach, a panel of inhibitors that target the downstream Raf-MEK-ERK kinases recently came into the focus as successful alternative approaches (Neuzillet al., 2014). Most MEK1/2 inhibitors exhibit reversible kinase inhibitor activity and relatively short half-lives (Wu et al., 2015; Gilmarten et al., 2011).

Systolic heart failure (HF) is characterized by exaggerated sympathetic nerve activity (SNA), which is a culprit behind further heart performance deterioration (Triposkiadis et al., 2009; Parati et al., 2012). The hypothalamic paraventricular nucleus (PVN) is a region in the forebrain rich in presympathetic neurons that regulate most neurohumoral responses related to sympathetic excitation (Yu et al., 2016; Yu et al., 2013). In HF, the neurochemical signals that control sympathetic activity in the PVN are deranged, with increased renin-angiotensin system (RAS) activity, endoplasmic reticulum (ER) stress, and elevated levels of proinflammatory cytokines (Wei et al, 2016; Wei et al., 2009; Wei et al., 2012; Zhang et al., 2012). Central interventions that interfere with these neurochemical abnormalities consistently inhibit SNA and improve the peripheral manifestations of HF.

### SUMMARY

In one embodiment, the disclosure provides an injectable MEK1/2 inhibitor-loaded microparticle formulation. In one embodiment, the formulation provides for sustained release of the MFK1/2 inhibitor. In one embodiment, the inhibitor comprises PD98059, SL327, pimasertib, cobimetinib, selumetinib, refametinib, trametinib, U0126, Ro 09-2210, CI-1040, PD0325901, RO4987655, RO5126766, bninmetinib, TAK733, GDC-0623, G-573, E6201, AS703988/MSC20150138, MEK-162, AZD-8330, TAK-733, GDC-0623, WX-554 or HL-085. In one embodiment, the microparticles have an average diameter of about 10 uni to about 25 μm or about 25 μm to about 50 μm.

PD98059 is a reversible MIX inhibitor that is a potential treatment for neurochemical changes in the brain that drive neurohumoral excitation in heart failure. PD98059 gains access to the brain to inhibit phosphorylation of ERK1/2 in the paraventricular nucleus of the hypothalamus, ultimately reducing sympathetic excitation which is a major contributor to clinical deterioration. Additional studies revealed that the pharmacokinetics of PD98059 matches a 2-compartment model with a short elimination half-life in plasma (approximately 73 minutes) that would severely limit its potential clinical usefulness.

To increase the availability of PD98059 to tissues, as described herein, a sustained-release PD98059-loaded microparticle formulation, e.g., a PLGA microparticle formulation, was prepared, using, for instance, an emulsion solvent evaporation technique. In one embodiment, the average particle size, yield percent, and encapsulation percent were found to be 16.73 μm, 76.6%, and 43%, respectively. in vitro drug release occurred over four weeks, with no noticeable burst release. Following subcutaneous injection of the microparticles in rats, steady plasma levels of PD98059 were detected by HPLC for up to two weeks. Furthermore, plasma and brain levels of PD98059 in rats with heart failure were detectable by LC./MS, despite expected erratic absorption. These findings suggest that a MEK1/2 inhibitor loaded microparticle, e.g., PD98059-loaded microparticles, may be employed as a therapeutic intervention to counter sympathetic excitation in heart failure, and perhaps in other disease processes, including cancers, in which activated MAPK signaling is a significant contributing factor.

In one embodiment, a method of preventing, inhibiting or treating sympathetic excitation or activated MAPK signaling, e.g., in heart failure or cancer, is provided. The method includes administering to a mammal in need thereof an effective amount of a composition comprising microparticles comprising a MEK1/2 inhibitor. In one embodiment, the MEK1/2 inhibitor is a reversible inhibitor. In one embodiment, the administration is local. In one embodiment, the administration is systemic. In one embodiment, the mammal is a human.

### BRIEF DESCRIPTION OF THE FIGURES

FIGS. 1A-1C. PD98059-loaded PLGA microparticles characterization. A) Particle size distribution. B) Morphology of the microparticles by SEM. C) Cumulative release of PD98059 from the microparticles compared to dissolution of PD98059 powder in the release medium (n=3, data represented are mean±SD).

FIG. 2. Western blot analysis of phosphorylated (p-) ERK1/2 levels in the PVN of HF rats two weeks after the SC injection of PD98059-loaded PLGA microparticles or blank microparticles. Top panel: Western blot bands. Bottom panel: values are represented as means±SEM (n=4-5/group, *p<0.05).

FIG. 3. Plasma norepinephrine levels in FIT rats two weeks after the SC injection of PD98059-loaded PLGA microparticles or blank microparticles. Values are represented as means d: SEM (n=4-5/group, *p<0.05).

FIG. 4. Schematic diagram describing the role of the PVN in the progression of heart failure, and how the long-term supply of the MEK1/2 inhibitor PD98059 using sustained release microparticles significantly mitigates p-ERK1/2 levels in the PVN. This results in a reduction in sympathetic nervous system activity, as represented by lower plasma norepinephrine levels.

FIGS. 5A-5D. Characterization of the prepared PD98059-loaded PLGA microparticles. A) Scanning electron microscopy (SEM) image of the drug-loaded microparticles. B) Particle size distribution, measured by ImageJ analysis of 100 particles in SEM images. C) Differential scanning calorimetry (DSC) thermograms of PD98059 (green), PLGA (black), a physical mixture of the two (blue), and PD98059-loaded microparticles (red). D) In vitro release profile of PD98059 from PLGA microparticles (data represent mean±SD, n=3).

FIGS. 6A-6B. A) Plasma PD98059 levels vs. time (n=3 for all time points, except 6 h, which has n=1) and B) organ levels of PD98059 (data represent mean±SD, n=3) following IV injection of 1 mg of PD98059 dissolved in 10% w/v Tween 80 aqueous solution in healthy rats

FIG. 7. Comparison of Plasma PD98059 levels vs. time curves following IV injection of PD98059 and SC injection of PD98059-loaded PLGA microparticles (data represent mean±SD, n=3). AUC₀₋ₜ calculations were based on non-compartmental analysis (NCA) using PK Solver ad-in.

FIG. 8. Plasma and brain PD98059 levels vs. time following the SC injection of 3.6 mg of PD98059 in PD98059-loaded. PLGA microparticles in rats with heart failure (data represent mean±SD, n=2-3).

FIG. 9. Pharmacokinetics of PD98059 following IV injection.

FIG. 10. Schematic of exemplary preparation and administration of microparticle formulation.

FIGS. 11A-11C. PD98059-loaded PLGA microparticles characterization. A) Particle size distribution. B) :Morphology of the microparticles by SEM. C) Cumulative release of PL98059 from the microparticles compared to dissolution of PD98059 powder in the release medium (n=3, data represented are mean±SD).

FIG. 12. Western blot analysis of pERK1/2 levels in the PVN of HF rats two weeks after the SC injection of PD98059-loaded PLGA microparticles or blank microparticles. Top panel: Western blot bands. Bottom panel: values are represented as means±SEM (n=4-5/group, *p<0.05).

FIG. 13. Plasma norepinephrine levels in HF rats two weeks after the SC injection of PD98059-loaded PLGA microparticles or blank microparticles. Values are represented as means±SEM (n=4-5/group, *p<0.05).

FIG. 14. Schematic diagram describing the role of the PVN in the progression of heart failure, and how the long-term supply of the MEK1/2 inhibitor PD98059 using sustained release microparticles may significantly mitigate p-ERK1/2 levels in the PVN. This results in sympathetic inhibition, as represented by lower plasma norepinephrine levels.

FIGS. 15A-15 D. A) SEM images of large PD98059-loaded PLGA microparticles. B) Particle size distribution of the microparticles. C) PD98059 release from the large PD98059-loaded PLGA microparticles (n=3, average±SD). D) Plasma levels following SC injection of the large PD98059-loaded microparticles in rats (n =3-4).

### DETAILED DESCRIPTION

Research over the past fifteen years has revealed that the Ras-Raf-MEK-ERK pathway in the PVN plays a role in the sympathetic excitation that accompanies, and ultimately aggravates, heart failure in rats. In heart failure, it was found that sympathetic excitation sympathetic nerve activity; SNA) that originates in the PVN by the action of the upregulated excitatory agonists takes place following activation of the downstream kinase ERK1/2 in the PVN . Higher levels of p-ERK1/2 were found in the PVN of HF rats, along with PVN neuronal excitation. PVN neuronal excitation and sympathetic nerve activity were inhibited by a short-term 1-hour intracerebroventricular (XV) infusion of PD98059, a specific MEK1/2 inhibitor, in heart failure rats. Long-term ICV infusion of PD98059 (for 4 weeks) normalized plasma levels of norepinephrine (NE) in heart failure rats, indicating that prolonged inhibition of p-ERK1/2 decreases sympathetic nerve activity. However, the short half-life and reversible mode of action of PD98059 are obstacles to achieving long-term activity in a clinical setting. While long term ICV infusion of the drug successfully decreased PVN p-ERK1/2, and subsequently plasma NE. levels in HF rats, the highly invasive nature of this procedure diminishes its clinical application.

The formulations disclosed herein provide for sustained release of MEK1/2 inhibitors, providing continuous exposure of brain structures to potentially therapeutic sustained plasma drug levels. In one embodiment, the highly selective MEK1/2 inhibitor PD98059 was used in such a formulation and was found to cross the blood brain hauler and to inhibit p-ERK1/2 levels in the PVN.

In particular, as disclosed herein, an alternative way to achieve long-term 2.5 PVN p-ERK1/2 inhibition was achieved in HIF rats, via subcutaneous (SC) injection of microparticles, e.g., sustained-release poly lactide-co-glycolide (PLGA) microparticles, loaded with PD98059. Two weeks after injection, PVN p-ERK1/2 levels were significantly decreased (p<0.05) compared to vehicle-treated HF rats. Circulating NE levels also decreased significantly. The formulation did not exhibit any noticeable toxicity in HF rats compared to untreated HF rats. These initial studies suggest that this approach for long-term control of central manifestations of HF may allow for efficacy, safety, and low frequency of administration, as a mono- or adjuvant therapy in combination with other pharmacological agents that act peripherally.

### Exemplary Formulations

In one embodiment, the formulation comprises particles comprising one or more MEK1/2 inhibitors. The disclosed particles, e.g., biodegradable microparticles, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and. Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed particles may be prepared by methods known in the art, (See, e.g., Nagavarma et al., Asian J. of Pharma. And Clin. Res., Vol 5, Suppl 3. 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing particles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the particles may be prepared by forming a double emulsion (e.g., water-in-oil-in-water) and subsequently performing solvent-evaporation. The particles may be subjected to further processing steps such as washing and lyophilization, as desired. Optionally, the particles may be combined with a preservative e.g., trehalose)

In one embodiment, the particles have a mean effective diameter of less than 500 microns, e.g., the particles have a mean effective diameter of between about 1 μm and about 500 μm, e.g., between about 5 μm and about 25 μm, about 10 μm and about 20 μm, about 15 μm and about 25 μm, about 100 μm to about 150 μm, or about 45 μm to 650 μm. In one embodiment, the particles have a mean effective diameter of less than 50 microns, e.g., the particles have a mean effective diameter of between about 0.01 μm and about 50 μm, e.g., between about 0.5 μm and about 5 μm, about 1 μm and about 10 μm, about 1 μm and about 7.5 μm, about 5 μm to about 10 μm, or about 2 μm to about 5 μm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, the particles comprise polymers including but not limited to polylactic-co-glycolic acid) (PLGA), polylactic acid (PLA), linear and/or branched PEI with differing molecular weights (e.g., 2, 22 and 25 kDa.), dendrimers such as polyamidoamine (PAMAM) and polymethoacrylates; lipids including but not limited to liposomes, emulsions, DOTAP, DOTMA, DMRIE, DOSPA, distearoylphosphatidylcholine (DSPC), DOPE, or DC-cholesterol; peptide based vectors including but not limited to poly-L-lysine or protamine; or poly(β-amino ester), chitosan, PEI-polyethylene glycol, PEI-mannose-dextrose, DOTAP-cholesterol or RNAiMAX.

In one embodiment, the particle is a glycopolymer-based particle, poly(glycoamidoamine)s (PGAAs). These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units may yield exceptional delivery efficiency.

In one embodiment, the particles comprise polyethyleneimine (PEI), polyamidoamine (PAMAM), PEI-PEG-, PEI-PEG-mannose, dextran-PEI, OVA conjugate, PLGA microparticles, or PLGA microparticles coated with PAMAM, or any combination thereof. The polymer may include, but is not limited to, polyamidoamine (PAMAM) dendrimers. Polyamidoamine dendrimers suitable for preparing the particles may include 3rd-, 4th-, 5th-, or at least 6th-generation dendrimers.

In one embodiment, the delivery vehicle may be particles or liposomes comprising a cationic lipid, e.g. N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N-(N,N′-dimethylaminoethane) carbamoyl] cholesterol (DC-Chol); dioctadecyl amidoglyceryl spermine (DOGS, Transfectam), or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et at, 2008; van der Woude et al., 1997; Ilies et al., 2004). In addition to pyridinium cationic lipids, other types of heterocyclic head group include imidazole, piperizine and amino acid. The main function of cationic head groups is to condense negatively charged molecules by means of electrostatic interaction to slightly positively charged particles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of de airliners include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although other materials, for example, Phi, DOTMA, DC-Chol, or CTAB, may be used.

In one embodiment, the particles comprise hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, polyethylene oxide)/poly(propylene oxide) block copolymers, polyethylene glycol)/poly(D,L-lactide-co-glycolide) block copolymers, polyglycolide, polylactides (PULA or PI)A), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and poly hydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e,g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), polylactic acid colysine) and polycaprolactone.

In one embodiment, the biocompatible material is derived from isolated extracellular matrix (ECM). ECM may be isolated from endothelial layers of various cell populations, tissues and/or organs, es., any organ or tissue source including the dermis of the skin, liver, alimentary, respiratory, intestinal, urinary or genital tracks of a warm blooded vertebrate. ECM may be from a combination of sources. Isolated ECM may be prepared as a sheet, in particulate form, gel form and the like.

The biocompatible polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, polyvinyl alcohol), silicone, hydrophobic polyesters and hydrophilic polyester, poly(lactide-co-glycolide), N-isoproylacrylamide copolymers, polyethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, polyethylene terephthalate), poly(tetrafluoroethylene), poly-epsilon-caprolactone, or crosslinked chitosan hydrogels.

### Pharmaceutical Compositions

The disclosure provides a composition comprising, consisting essentially of, or consisting of microparticles, nanoparticles or liposomes comprising one or more MEK1/2 inhibitors and optionally a pharmaceutically acceptable (e.g., physiologically acceptable) carrier. In one embodiment, additional components can be included that do not materially affect the composition (e,g., adjuvants, buffers, stabilizers, anti-inflammatory agents, solubilizers, preservatives, etc.). In one embodiment, when the composition consists of the polymer or particles formed therefrom, the inhibitor and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of, in one embodiment, the MEK1/2 inhibitor encapsulated in particles. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the MEK1/2 inhibitor is administered in a composition formulated to protect the MEK1/2 inhibitor from damage prior to administration. In addition, one of ordinary skill in the art will appreciate that the MEK1/2 inhibitor can be present in a composition with other therapeutic or biologically-active agents.

Injectable depot forms are envisioned including those having biodegradable polymers such as polylactide-polyglycolide. Depending on the ratio of inhibitor to polymer, and the nature of the particular polymer employed, the rate of inhibitor release can be controlled. Examples of other biodegradable polymers include poly(orthoesters) and poly(anhydrides). Depot injectable formulations are also prepared by entrapping the inhibitor optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the MEK1/2 inhibitor in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the MEK1/2 inhibitor to elicit a desired response in the individual. One of ordinary skill in the art can readily determine an appropriate MEK1/2 inhibitor dose range to treat a patient having a particular disease or disorder, based on these and other factors that are well known in the art.

In one embodiment, the composition is administered once to the mammal. It is believed that a single administration of the composition may result in persistent expression in the mammal, optionally with minimal side effects. However, in certain cases, it may be appropriate to administer the composition multiple times during a therapeutic period to ensure sufficient exposure of cells to the composition. For example, the composition may be administered to the mammal two or more times (e.g., 4, 5, 6, 6, 8, 9, or 10 or more times) during a therapeutic period.

The present disclosure provides pharmaceutically acceptable compositions which comprise a therapeutically-effective amount of the MEK1/2 inhibitor as described above.

### Routes of Administration Dosages and Dosage Forms

Administration of the MEK1/2 inhibitor may be continuous or intermittent, depending, for example, upon the recipient's physiological condition, and other factors known to skilled practitioners. The administration of the MEK1/2 inhibitor may be essentially continuous over a preselected period of time or may be in a series of spaced doses. Both local administration, e.g., intranasal or intrathecal, and systemic administration are contemplated. Any route of administration may be employed, e.g., intravenous, intranasal or intrabronchial, or local administration. In one embodiment, compositions may be subcutaneously, orally or intravascularly delivered.

One or more suitable unit dosage forms comprising the MEK1/2 inhibitor, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the MEK1/2 inhibitor with liquid carriers, solid matrices, semi-solid carriers, finely divided solid carriers or combinations thereof, and then, if necessary, introducing or shaping the product into the desired delivery system.

The amount of the MEK1/2 inhibitor administered to achieve a particular outcome will vary depending on various factors including, but not limited to the condition, patient specific parameters, e.g., height, weight and age, and whether prevention or treatment, is to be achieved.

The MEK1/2 inhibitor may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The MEK1/2 inhibitor may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The MEK1/2 inhibitor can be provided in a dosage form containing an amount effective in one or multiple doses. The MEK1/2 inhibitor may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 phosphate buffered saline. In one embodiment, from 0.0001 to 1 mg or more, e.g., up to 1 g, in individual or divided doses, e.g., from 0.001 to 0.5 mg. or 0.01 to 0.1 mg, of MEK1/2 inhibitor can be administered.

Pharmaceutical formulations containing the MEK1/2 inhibitor can be prepared by procedures known in the art using well known and readily available ingredients. For example, the agent can be formulated with common excipients, diluents, or carriers, and formed into tablets, capsules, suspensions, powders, and the like. The MEK1/2 inhibitor can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the MFK1/2 inhibitor may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the MEK1/2 inhibitor composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the MEK1/2 inhibitor composition may be administered via nose drops, a liquid spray, such as via a plastic bottle atomizer or metered-dose inhaler. Typical of atomizers are the Mistometer (Wintrop) and the Medihaler (Riker).

The local delivery of the MEK1/2 inhibitor composition can also be by a variety of techniques which administer the MEK1/2 inhibitor composition at or near the site of disease, e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary Embodiments

In one embodiment, a composition comprising microparticles comprising one or more MEK1/2 inhibitors is provided. In one embodiment, the inhibitor is a reversible inhibitor. In one embodiment, the composition is injectable. In one embodiment, the composition provides for sustained release of the inhibitor, e.g., over 1 to 2 weeks, over 2 to 4 weeks, or over 1 to 3 months. In one embodiment, the diameter of the microparticles is about 1 μm to about 10 μm, about 10 μm to about 50 μm, about 10 μm to about 25 μm, about 2 μm to about 6 μm, about 50 μm to about 200 μm, or about 200 to about 400 μm. In one embodiment, the microparticles are biocompatible and biodegradable. In one embodiment, the microparticles are formed of a polyester. In one embodiment, the microparticles are formed of a natural polymer. In one embodiment, the microparticles are formed of lactic acid, glycolic acid, or combinations thereof. In one embodiment, the inhibitor comprises PD98059, SL327, pimasertib, cobimetinib, selumetinib, refametinib, trametinib, U0126, Ro 09-2210, CI-1040, PD0325901, RO4987655, RO5126766, binimetinib, TAK733, GDC-0623, G-573, E6201, or AS703988/MSC20150138.

In one embodiment, a method to prevent, inhibit or treat heart failure in a mammal is provided comprising administering to the mammal an effective amount of the composition. In one embodiment, the mammal is a human. In one embodiment, the composition is injected. In one embodiment, the composition is locally administered. In one embodiment, the composition is systemically administered. In one embodiment, the composition is intravenously administered. In one embodiment, the composition is subcutaneously administered. In one embodiment, the microparticles are formed of a natural polymer. In one embodiment, the microparticles are formed of lactic acid, glycolic acid, or combinations thereof. In one embodiment, the inhibitor comprises PD98059, SL327, pimasertib, cobimetinib, selumetinib, refametinib, trametinib, U0126, Ro 09-2210, CI-1040, PD0325901, RO4987655, RO5126766, binimetinib, TAK733, GDC-0623, G-573, E6201, or AS703988/MSC20150138. In One embodiment, the microparticles are PLGA microparticles, e.g., having lactide:glycolide ratio of 30:70, 40:60, 45:55, 50:50, 60:40, 55:45, or 70:30. In one embodiment, the microparticles formed of a polymer having a Mw of about 24,000 to about 38,000. In one embodiment, the microparticles formed of a polymer having a Mw of about 7,000 to about 17,000. In one embodiment, the microparticles formed of a polymer having a Mw of about 20,000 to about 40,000. In one embodiment, the microparticles formed of a polymer having a Mw of about 5,000 to about 20,000.

In one embodiment, a method to prevent, inhibit or treat sympathetic nerve activation in a mammal is provided comprising administering to the mammal an effective amount of the composition. In one embodiment, the mammal has cancer. In one embodiment, the microparticles are formed of a natural polymer. In one embodiment, the microparticles are formed of lactic acid, glycolic acid, or combinations thereof. In one embodiment, the inhibitor comprises PD98059, SL327, pimasertib, cobimetinib, selumetinib, refametinib, trametinib, U0126, Ro 09-2210, CI-1040, PD0325901, RO4987655, RO5126766, binimetinib, TAK733, GDC-0623, G-573, E6201, or AS703988/MSC20150138.

The invention will further be described by the following non-limiting examples.

### EXAMPLE 1

### Introduction

PD98059 was chosen as an exemplary MEK1/2 inhibitor because it was found to cross the blood brain barrier (BBB) to reach the PVN (sequestered behind the BBB) at therapeutic levels. The short half-life of PD98059 and its reversible MEK1/2 inhibition activity are the two major hurdles that impede the progress of the application of PD98059 in the clinic. The elimination half-life of PD98059 following IV injection of a solution of the drug in rats was around 70 min. Earlier, Dudley et al. reported that the in vitro activity of MEK was fully and instantaneously restored once PD98059 was removed from the medium.

Additionally, previous data showed that 1-h ICV infusion significantly decreased heart rate in HF rats (compared to SHAM or untreated animals)

### Materials

PD98059 was purchased from Selleck Chemicals (Houston, Tex.). Poly (lactide-co-glycolide) (PLGA, Resomer RG 503) was purchased from Evonik (Parsippany, N.J.). Poly vinyl alcohol (Mowiol 8-88, MW 67,000) was purchased from Sigma Aldrich (St Louis, Mo.). Tween 80 was purchased from Fisher Chemicals (Waltham, Mass.). All other chemicals were of analytical grade and used without further purification.

### Microparticles Preparation

Microparticles were prepared using single emulsion solvent evaporation technique. Briefly, 2.75 mg of PD98059 were dissolved in 300 ul of dichloromethane (DCM). Then, 200 mg of PLGA were also dissolved in 1.2 ml of DCM, and the two solutions were combined in a single vial. This organic solution (oil phase) was added to an aqueous phase composed of 30 ml of 1% w/v polyvinyl alcohol (PVA) in 150 ml beaker. The mixture was homogenized (Ultra-turrax T25 basic, Ika Works Inc., Wilmington, N.C.) at 17500 rpm for 30 seconds. Solvent evaporation was achieved following stirring of the emulsion at room temperature at 600 rpm for 90 minutes under a fume hood. The microparticles were collected by centrifugation at 1000×g for 5 min (Eppendorf Centrifuge 5864 R, Eppendorf, Hauppauge, N.Y.) and the supernatant was rejected. The microparticles were washed twice in 30 ml Nano-Pure water (Barnstead Thermolyne Nanopure water. Thermo Fisher, Waltham, Mass.) followed by centrifugation. The microparticles were then resuspended in 5 ml of Nano-Pure water, frozen at −80° C., and lyophilized overnight (Labconco Free zone 4.5, Labconco, Kansas City, Mo.).

### Microparticle Characterization

### Microparticle Morphology

Scanning electron microscopy (SEM) was used to examine the microparticles morphology. A thin sheet of the lyophilized microparticles on a carbon double-tape mounted on an aluminum SEM stub was sputter-coated with gold and palladium (Emitech K550 sputter-coater). The SEM images were taken using Hitachi S-4800 scanning electron microscope (Hitachi High Technologies America Inc., Schaumburg, Ill.). The particle sizes of about 70 particles in SEM images were measured using ImageJ (NIH, Bethesda, Mass.) and the data were plotted using Microsoft Excel.

### Drug Content Determination

A weighed amount of the microparticles was dissolved in DCM at a concentration of 1 mg/ml. One hundred microliters of this solution were diluted with methanol to 6.5 ml, and finally centrifuged at 12,000×g for 5 min. The supernatant was further diluted with purified water to 10 ml, and the concentration of the resultant solution was measured by HPLC as mentioned below.

Drug content (μg/mg) of the microparticles was calculated using equations 1, as follows:

$$Drug content ( \frac{μg}{mg} ) = \frac{amount of PD98059 ( μg )}{weight of microparticles after lyophilization ( mg )} \qquad Equation 1$$

### In Vitro Drug Release

A weighed amount of the microparticles was suspended in 5 ml of the release medium (Dulbecco's phosphate buffered saline, DPBS, Life Science, Waltham, Mass.) that contains 0.4% w/v Tween 80 at an amount equivalent to 0.065 mg microparticles in 50 ml tube (n=3). The tubes were placed in an orbital shaker (New Brunswick Scientific, Edison, N.J.) operating at 300 rpm and 37° C. At pre-determined time points, the tube was centrifuged (1000×g for 5 min) and the whole volume of the release medium (5 ml) was removed and replaced completely with fresh medium in which the pellet was re-suspended. The concentration of PD98059 was measured in the samples using the HPLC method described below. The standard curve range was 0.125-10 μg/ml, and the r² value was 0.9998.

### Experimental Protocol

Nine Male Sprague-Dawley rats (6-8 weeks) weighing about 275-330 g, obtained from Envigo. Indianapolis, Ind. were used in the experiment and were kept at the University of Iowa animal care facility. They were kept under controlled temperature at around 23±2° C. and were exposed to 12-hours of light and dark cycles. Food was provided to the rats ad All animal experiments performed were approved by the University of Iowa Institutional Animal Care and Use Committee.

Heart failure in anesthetized rats (ketamine/xylazine) was induced by ligation of the left coronary artery under sterile conditions. Twenty four hours later, heart failure was confirmed by echocardiography in the form of reduced systolic function (with rats showing left ventricular ejection fraction of less than 40%), then the microparticles suspension (PD98059-loaded or blank, n=4-5/group) was injected SC in the rats. The microparticles suspension for SC injection was prepared by suspending an accurately weighed amount of lyophilized PD98059-loaded microparticles (containing 0.4 mg of PD98059/rat), or an equal amount of the blank microparticles, in 1 ml of 1× DPBS and injected SC in the shaved back of each rat.

Two weeks after the microparticles injection, the HF rats were euthanized by decapitation following urethane anesthesia, then their brains were collected, and total ERK1/2 and p-ERK1/2 levels were determined by Western blot analysis in PD98059-loaded microparticles rats (n=5), and blank microparticles rats (n=4). Protein levels of total ERK1/2, and p-ERK1/2, and β-actin were analyzed by Western blot analysis using primary antibodies to p-ERK1/2 and β-actin (Cell Signaling Technology, Dartvers, Mass.). The bands densities were quantified using Image Lab analysis software (Bio-Rad, Hercules, Calif.).

Plasma norepinephrine (NE) levels in HF rats (n=5 for PD98059-loaded microparticles rats and n=4 for blank microparticles rats) were measured by an ELISA kit (Rocky Mountain Diagnostics, Colorado Springs, Colo.) according to the manufacturer's instructions.

### HPLC

An Agilent Infinity 1100 HPLC (Santa Clara, Calif.) was used for to analyze PD98059 content and release of the microparticles. The HPLC consisted of a quaternary pump (Agilent Technologies), diode array detector (DAD, Agilent Technologies) and auto-injector (Agilent Technologies). A Waters Symmetry Reversed phase C-18 (RP-C18) column was used for PD98059 assay (5 μm, 4.6 mm×150 mm, Milford, Mass.). The mobile phase composition was methanol:water 70:30 with 0.1% v/v trifluoroacetic acid (TFA). The flow rate used was 1 ml/min at room temperature, and the wavelength at which PD98059 was detected at 275 nm.

### Statistical Analysis

All data presented are means±SD. Statistical significance was analyzed by one-way ANOVA followed by Tukey's post-hoc test or Student T-test. Data were considered significant if p value is <0.5.

### Results

A single injection of a sustained-release dosage form of PD98059, e.g., PD98059-loaded biodegradable microparticles, was able to significantly decrease the elevated levels of p-ERK1/2 in the PVN and NE in plasma of HF rats for up to 2 weeks.

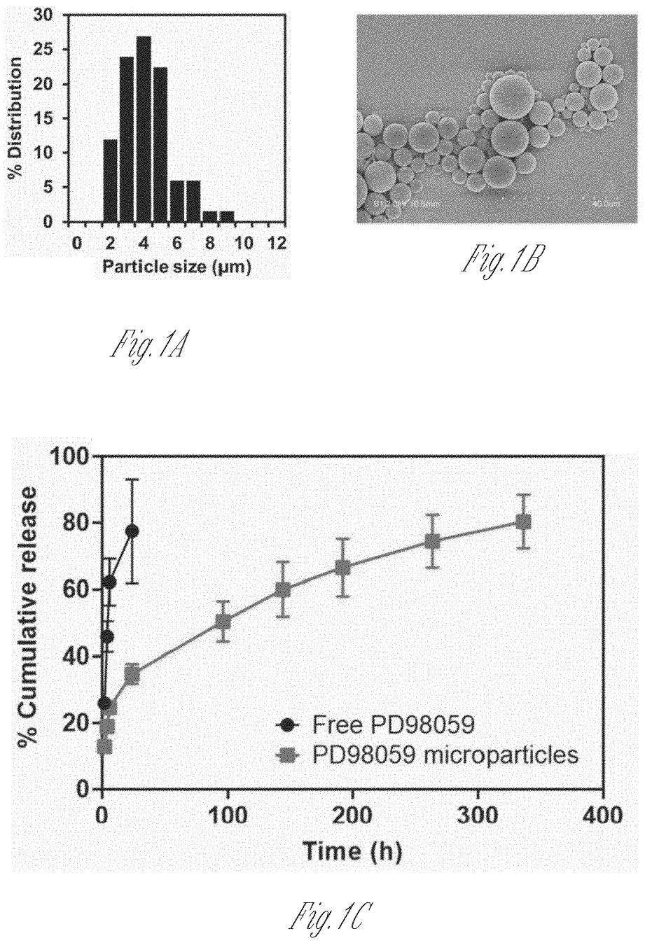

Poly lactide-co-glycolide (PLGA, Resomer RG502, lactide: glycolide ratio of 50:50, molecular weight 24-38 kDa, Evonik, Birmingham, Ala.), a biodegradable polyester that degrades in vivo into biocompatible by-products, was used to prepare the microparticles. The microparticles were spherical and had smooth surface, as can be seen in the scanning electron microscopy image (FIG. 1a). The average particle size (FIG. 1b) and drug loading of these particles were found to be around 4 μm and 13.22+1.24 μg drug per mg microparticles, respectively. The microparticles exhibited slow drug release, as approximately 60% of the loaded drug was released in the first week, and about 80% were released after two weeks. Meanwhile, it took only 24 hours for 80% of the unencapsulated drug to dissolve in the release medium. This shows the ability of the microparticles to control the drug release in vitro.

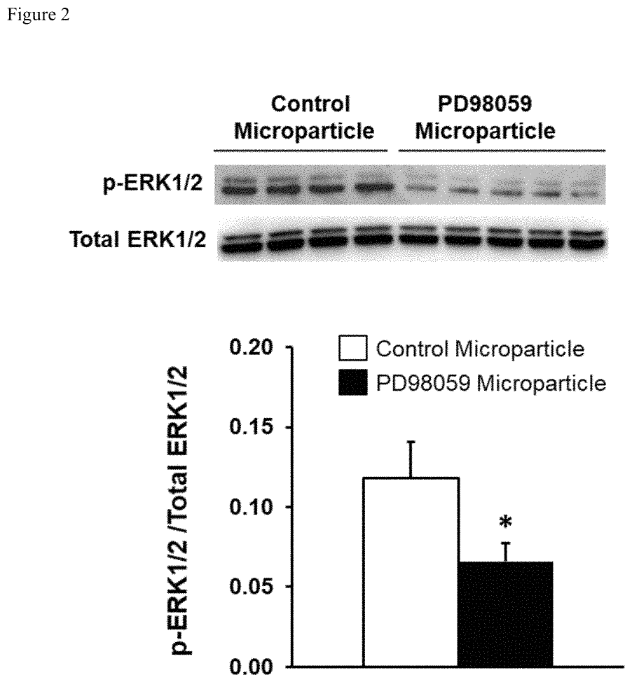

Heart failure was induced in rats by ligation of the left coronary artery. Heart failure was confirmed by echocardiography, then microparticles were injected SC at a dose of 400 μg per rat in 1 ml of Dulbecco's phosphate buffer saline (DPBS, pH 7.4, Thermo Fisher, Waltham, Mass.). Two weeks after microparticle injection, p-ERK1/2 levels in the PVN (normalized to total ERK1/2) in rats treated with PD98059-loaded microparticles were found to be about half of those in rats treated with blank microparticles (p<0.05, FIG. 2).

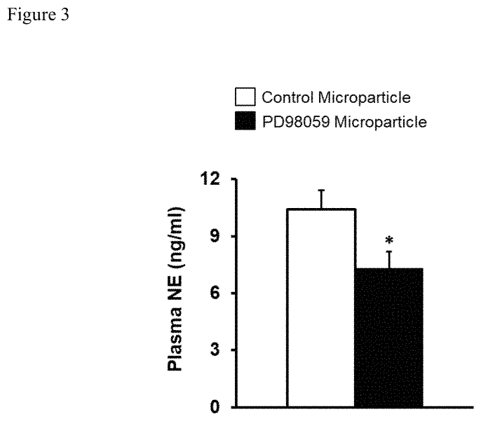

It was determined whether this inhibition of p-ERK1/2 in the PVN was reflected systemically. Plasma norepinephrine (NE) level was used as a general indicator of sympathetic activity. Circulating NE level decreased significantly two weeks after SC injection of PD98059-loaded microparticles in HF rats, compared to HF rats injected with empty PLGA microparticles (FIG. 3). Previously it was shown that a significant reduction of NE plasma levels was achieved following the chronic treatment of HF rats with ICV infusion of the MEK1/2 inhibitor PD98059, and to a lesser extent the P38 IN/IAPK inhibitor SB203580 for 4 weeks. This was not achieved when the c-Jun N-terminal kinase inhibitor SP600125 was given to HF rats in the same manner.

No evidence of toxicity or adverse events was found following the SC injection of these microparticles in HF rats. Echocardiographic investigation did not reveal any negative effect on the left ventricular ejection fraction (LVEF). There was also no kidney toxicity, as outlined by detecting cystatin C levels and kidney injury molecule 1 (KIM-1) mRNA levels, and no liver toxicity as found by detecting aspartate aminotransferase (AST), alanine aminotransferase (ALT), and bilirubin serum levels compared to HF rats with no treatment. It was also found that the body weight was not altered, and no behavioral changes were noticed for two weeks after the injection

Congestive heart failure is a major cause of peripheral edema, which, in addition to poor perfusion, are expected to adversely affect extravascular drug absorption. The data showed that the microparticles were able to provide therapeutic levels of PD98059 to the brain in spite of poor perfusion that may hinder the drug's absorption.

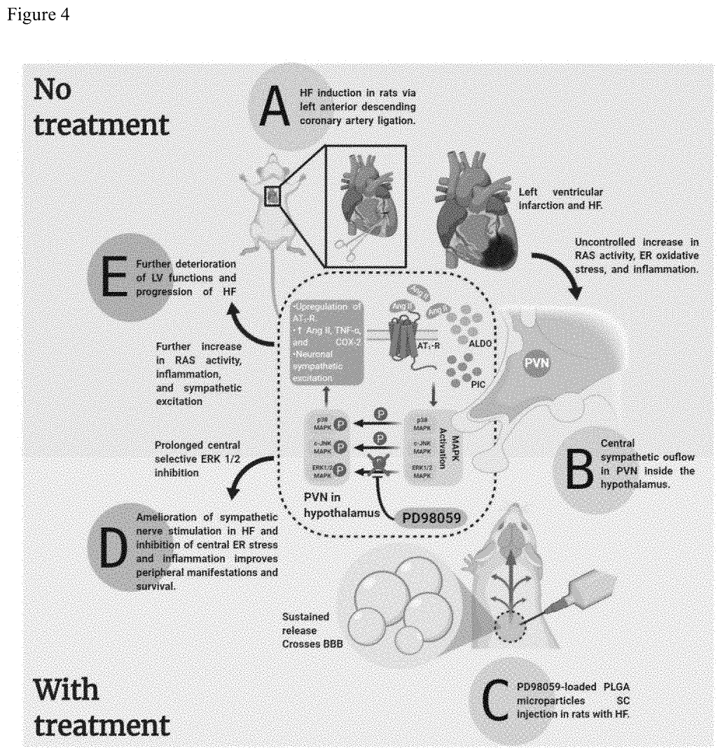

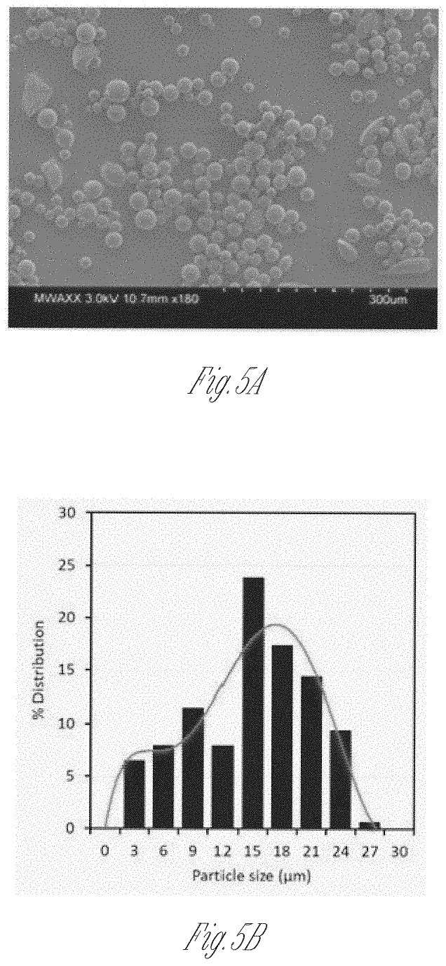

In summary, a sustained release formulation of a MEK1/2 inhibitor curbed the sympathetic excitation in HF rats for up to two weeks. This was achieved at least in part by prolonged inhibition of p-ERK1/2 in the PVN, the brain center in which deranged neurochemical signaling contributes to sympathetic overactivity in HF. The MEK1/2 inhibitor PD98059 was slowly released over 2 weeks at levels sufficient to suppress p-ERK1/2 levels in the PVN (FIG. 4). This had previously only been achieved using ICV administration, a highly invasive, clinically non-feasible approach. Two problems associated with PD98059 were successfully overcome using this approach; its short half-life and it reversible activity. Thus, the present formulations may be useful for long-term therapy that provides prolonged inhibition of p-ERK1/2 in the PVN, in theory for up to 3 months. From the clinical point of view, this approach, as a single therapy or an adjuvant therapy with other peripheral treatments (e.g., angiotensin converting enzyme (ACE) inhibitors, or β-blockers) to control increased sympathetic activity in HF, is desirable.

### EXAMPLE 2

### Introduction

Over the past decade, the role of the MAPK pathway in the PVN in regulating the sympathetic excitation in a rat model of heart failure-induced by myocardial infarction has been investigated. Initial studies revealed that p-ERK1/2 was increased in the PVN of rats with chronic heart failure, along with PVN neuronal activation, and that a 1-hour ICV infusion of the MEK1/2 inhibitor PD98059 decreased PVN neuronal excitation and renal sympathetic nerve activity in rats with heart failure. In subsequent work, chronic (4 week) ICV infusions of PD98059 in heart failure rats reduced plasma norepinephrine, an index of overall sympathetic nerve activity. These powerful effects of ICV PD98059 likely reflect the central interactions between ERK1/2 and major neurochemical systems in brain that drive sympathetic activity, including the brain renin-angiotensin system, neuroinflammatory cytokines and chemokines, and endoplasmic reticulum stress. However, because of its short half-life and its reversible inhibition of MEK1/2, PD98059 requires persistent drug exposure to be effective. Continuous drug administration is not practical and feasible clinically. In an effort to harness the therapeutic potential of PD98059 as an agent targeting central sympatho-excitatory mechanisms in heart failure, a pharmaceutical preparation was prepared that would deliver the drug to maintain a sustained plasma level sufficient to facilitate passage of effective levels of PD98059 into the brain.

### Materials

PD98059 was purchased from Selleck Chemicals (Houston, Tex.). Poly (lactide-co-glycolide) (PLGA., Resomer RG 503 H) was purchased from Evonik (Parsippany, N.J.). Poly vinyl alcohol (PVA, Mowiol 8-88, MW 67,000) and 7-hydroxyflavone were purchased from Sigma Aldrich (St Louis, Mo.). Tween 80 was purchased from Fisher Chemicals (Waltham, Mass.). All other chemicals and reagents were at least of analytical grade and were used as received without further purification.

### Preparation of the Microparticles

Microparticles were prepared using an emulsion-solvent evaporation method. Briefly, 200 mg of PLGA and 12 mg of PD98059 were dissolved in 1.5 ml of dichloromethane (DCM), and this organic solution was added into 30 ml of 1% PVA solution. The mixture was emulsified at 6500 rpm at room temperature for 5 min (Ultra-turrax T25 basic, Ika Works, Inc., Wilmington, N.C.). The emulsion was then magnetically stirred at room temperature at 600 rpm for 2 hours to evaporate DCM. The microparticle suspension was then collected by centrifugation at 1000×g for 10 min (Eppendorf Centrifuge 5864 R, Eppendorf North America, Hauppauge, N.Y.). The microparticles were resuspended in 45 ml of Nanopure water (Barnstead Thermolyne ⁻Nanopure water purification system. Thermo Fisher, Waltham, Mass.), washed and centrifuged as mentioned earlier. This process was carried out twice to remove any remaining PVA and unencapsulated PD98059. Finally, the microparticles were resuspended in 1 ml of purified water and lyophilized overnight at 0.045 mbar and a collector temperature of −105° C. (Labconco Free zone 4.5^{-105° C.}, Labconco, Kansas City, Mo.).

### In Vitro Characterization of the Prepared Microparticles

### Morphology of the Microparticles

The morphology of the microparticles was investigated using scanning electron microscopy (SEM). Microparticles (lyophilized) were spread onto a carbon double-adhesive tape mounted on an aluminum stub, and then were sputter-coated with gold and palladium using an argon beam Emitech K550 sputter-coater. A Hitachi S-4800 scanning electron microscope (SEM) operated at 3 kV accelerating voltage (Hitachi High Technologies America Inc., Schaumburg, Ill.) was used to capture the images of the microparticles. The particle size was analyzed using Image software (NIH, Bethesda, Mass.) after a minimum of 100 particles in SEM images were measured, and the data were plotted using Microsoft Excel Determination of microparticles drug content

Microparticles were dissolved in DCM at 1 mg/ml, then 100 μl of this solution was added to 6.4 ml of methanol, and centrifuged (12,000×g for 5 min). The supernatant was mixed with 3.5 ml of purified water, and the resultant solution was injected into the HPLC as mentioned below.

Drug content in the microparticles was calculated using equation 1, as follows:

$$Drug content ( \frac{μg}{mg} ) = \frac{amount of PD98059 ( μg )}{weight of microparticles after lyophilization ( mg )} \qquad Equation 1$$

Yield percentage was calculated using equation 2, as follows:

$$Yield \% = \frac{Weight of the lyophilized microparticles}{Weight of the starting particles} \times 100 \qquad Equation 2$$

Finally, encapsulation efficiency percentage (EE%) was calculated using equation 3, as follows:

$$EE \% = \frac{Amount of PD98059 in 1 mg of microparticles}{Expected amount of PD98059 in 1 mg of microparticles assuming 100 \% enc \text{?} \times 100} \\ \text{?} \text{indicates text missing or illegible when filed} \qquad Equation 3$$

The expected amount of PD98059 in 1 mg of microparticles, assuming 100% encapsulation, is 12 mg/212 mg (56.6 μg).

### Differential Scanning Calorimetry

Weighed amounts of PD98059, PLGA, PLGA/PD98059 physical mixture (20:1 w/w), and PD98059-loaded PLGA microparticles were added into aluminum crimped pans and differential scanning calorimetric (DSC) thermograms were obtained using a TA Instruments model Q20 DSC (New Castle, Del., USA). A temperature ramp rate of 5° C./min, within a range of 0 to 200° C. was used.

### In Vitro Drug Release

A weighed amount of the microparticles was suspended in 1× DPBS (Dulbecco's phosphate buffered saline, Life Science, Waltham, Mass.) at 0.5 mg microparticles/ml. One ml of this suspension was transferred to a 1 ml screw-capped dialysis tube (Spectra/Por™ Float-A-Lyzer™ G2 MWCO 8-10 kDa, Sigma-Aldrich). The tube was submerged in 12 ml of 0.4% v/v solution of Tween 80 in 1× DPBS and placed in an orbital shaker (New Brunswick Scientific, Edison, N.J.) at 300 rpm and 37° C. The solubility of PD98059 in this release medium was approximately 113 μg/ml at 37° C. At pre-determined time points, the whole volume of the release medium (12 ml) was removed and replaced completely with fresh medium. The concentration of PD98059 was measured in the samples using the HPLC method described below.

### Experimental Protocols

Twenty three male Sprague-Dawley rats (6-8 weeks, 275-300 g, Harlan labs, Indianapolis, Ind.) were used in this experiment. Rats were kept under controlled temperature (23±2° C.) at the University of Iowa animal care facility. They were exposed to 12-hours of light and dark cycles, and food was provided ad libitum. All animal experiments performed were approved by the University of Iowa Institutional Animal Care and Use Committee.

1: Pharmacokinetics of PD98059. Rats were anaesthetized using urethane and a canula was inserted in the femoral vein. A solution of PD98059 dissolved in 10% Tween 80 in sterile 1× DPBS at a concentration of 0.5 mg/ml (total volume 2 ml) was administered slowly by intravenous infusion. After 5, 15, 30, 60, 180, and 360 minutes, blood samples were withdrawn from rats. Major organs (liver, kidney, brain, and heart) were collected from rats after 1 and 3 hours (n=3/time point).

2: Subcutaneous Administration of PD98059-loaded PLGA Microparticles—Normal Rats. PD98059-loaded microparticles suspended in sterile 1× DPBS were injected subcutaneously (SC) in healthy rats (n×3) at a dose of 2.4 mg PD98059 in 1 ml per rat. At predetermined time intervals (30 min, 1 day, 7 days, and 14 days, 21 days, and 28 days), rats were anesthetized using isoflurane and blood samples were withdrawn from the tail vein. Plasma drug levels were determined by HPLC.

3: Subcutaneous Administration of PD98059-loaded PLGA Microparticles—Heart Failure Rats. Under sterile conditions, male Sprague-Dawley rats were anesthetized with ketamine/xylazine and underwent left coronary artery ligation to induce heart failure. Twenty-four hours later, and after heart failure was confirmed by echocardiographic demonstration of reduced systolic function (left ventricular ejection fraction <40%), the rats were subcutaneously injected with the microparticle suspension at a dose of 3.6 mg in 1 ml sterile PBS. After 1, 7, and 14 days, rats were euthanized (n=2-3 per time point) and their plasma and brains were collected.

### Plasma and Tissue Preparation.

All blood samples were collected into 4 ml BD Vacutainer® blood collection tubes (K2-EDTA, Becton, Dickinson, and Company, Franklin Lakes, N.J.). Plasma samples were collected by centrifugation (3,300×g, 15 min), and frozen at −80° C. until analyzed. Plasma samples were thawed on ice, and a volume of 100 μl of plasma was transferred to a 15 ml tube, and spiked with 15 μl of the internal standard (IS) solution (10 μg/ml of 7-hydroxyfiavone in methanol). Collected organs were rinsed in PBS, then frozen at −80° C. For organs, a portion of each organ (200-400 mg) was accurately weighed and homogenized (Fisher Brand Bead Mill 4 Homogenizer, Hampton, N.H.) in 250 μl of 1× DPBS using 20-25 2.5 mm ceramic beads per sample, and spiked with 15 μl of the internal standard (IS) solution. One ml of cold acetonitrile was added to 100 μl of plasma samples or 400 μl of the tissue homogenate and vortexed for 1 min. The samples were kept on ice for 15 min to precipitate the proteins, then the tubes were centrifuged (4° C., 3,300×g, 10 min) and the supernatant was collected, and evaporated under nitrogen stream.

In the case of protocols 1 and 2, the residue in each tube was then dissolved in the mobile phase (described in the HPLC section below), centrifuged (12,000×g, 5 min), and the supernatant was injected in the HPLC and analyzed by the method described below. A standard curve was prepared using plasma and tissues collected from naive rats. These plasma or tissue homogenate samples were spiked with 15 μl of the IS solution as mentioned above, in addition to 15 μl of standard solutions of PD98059 in methanol at different concentrations. Pharmacokinetic parameters were calculated using PK Solver.

In the case of protocol 3, residues were redissolved in the mobile phase and PD98059 levels in the plasma and brain were measured using LC/MS/MS with multiple reaction monitoring (MRM) using 7-hydroxytlavone as an IS. Standard curves were constructed in plasma and brain tissues collected from naive rats. HPLC and LC/MS/MS

An Agilent HPLC workstation was used for sample analysis (Agilent Infinity 1100, Santa Clara, Calif.) that consisted of an Agilent quaternary pump, automatic injection port, and Agilent diode array detector (Agilent Corporation, Santa Clara, Calif.). A RP-C18 column was used for analysis (Waters Symmetry, 5 μm pore size, 4.6 mm×150 mm, Milford, Mass.). The mobile phase consisted of methanol: water 70:30 with 0.1% v/v trifluoroacetic acid, and the flow rate was 1 ml/min at room temperature. The detection wavelength was set to 275 nm.

The LC/MS/MS system consisted of a Waters Acquity TQD (Milliford, Mass.), which includes a triple quadruple mass spectrometer and Acquity H-Class UPLC. The same column, temperature, mobile phase, and flow rate stated above with the HPLC method were used. Quantitative analysis of PD98059 and IS was carried out using positive electrospray ionization via the highly sensitive and specific MRM mode. PD98059 was detected at 3 transition channels for brain samples (268.03→104.86, 268.03→121.01, and 268.03→133.06) and 5 transition channels for plasma samples (268.03→104.86, 268.03→121.01, 268.03→133.06, 268.03→148.08, and 268.03→236.07), while the IS was detected as 3 transition channels in both brain and plasma samples (239.03→77.04, 239.03→129.03, 239.03→136.97). The standard curves were linear over a range of 0.1-30 μg/ml for both plasma and brain.

### Results

### Preparation and In Vitro Characterization of the Microparticles

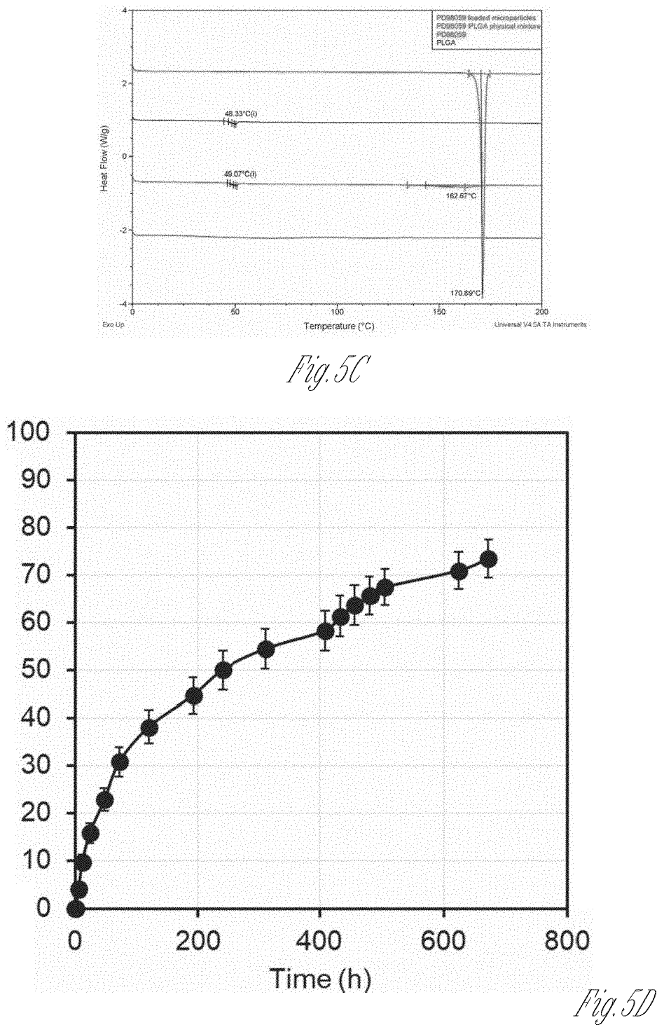

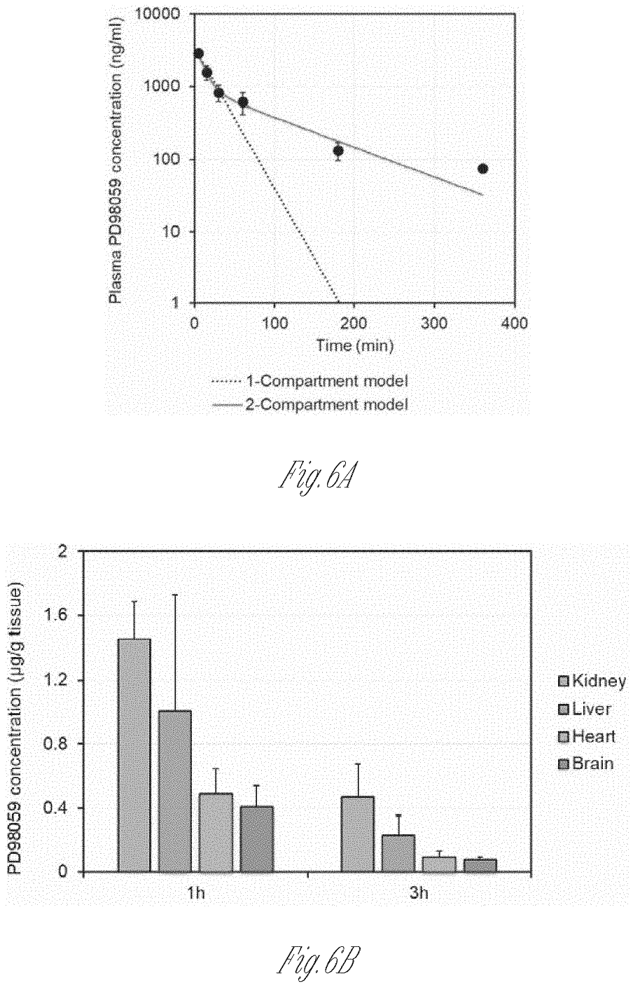

The drug-loaded microparticles contained 24.3 μg PD98059/mg. Scanning electron microscopy showed that the microparticles were mostly spherical in shape, with smooth surfaces, while no unencapsulated drug was observed (FIG. 5a), as observed when larger amounts of the drug were loaded. Particle size analysis revealed a normal distribution (FIG. 5b). Average particle size was approximately 16.7 μm (FIG. 5b). DSC thermograms (FIG. 5c) showed that the drug exhibited a sharp endothermic peak at 171° C. that indicates the drug melting point. The polymer had a brief endothermic peak at 48.33° C., which indicates the glass transition temperature (T_{g}) of the polymer. The physical mixture of the drug and polymer at a ratio of 1:20 showed a T_{g} of PLGA (at 49.1° C.), while the drug melting point peak appeared as a broad endothermic incident at 162.3° C. The microparticles did not show any endothermic peaks, neither at the polymer T_{g}, nor at or around the melting point of PD98059. This may indicate physicochemical interaction between PD98059 and PLGA, which resulted from the drug being in an amorphous state, or may simply be due to the microparticle preparation processing. The average particle size, yield %, drug loading (μg drug/mg microparticles), and encapsulation efficiency % (EE %) are 16.73±6.22 μm, 76.6±2.35%, 24.33±3.1 μg/mg, and 43±5.47%, respectively. In vitro drug release from PLGA microparticles was slow, with less than 40% of loaded drug being released in the first week. Approximately 73% of the loaded drug was released within four weeks (FIG. 5d). In general, the release followed a biphasic pattern with no burst release, where the initial release during the first two days was faster than the rest of the release period.

### Pharmacokinetics of PD98059,Following IV Injection

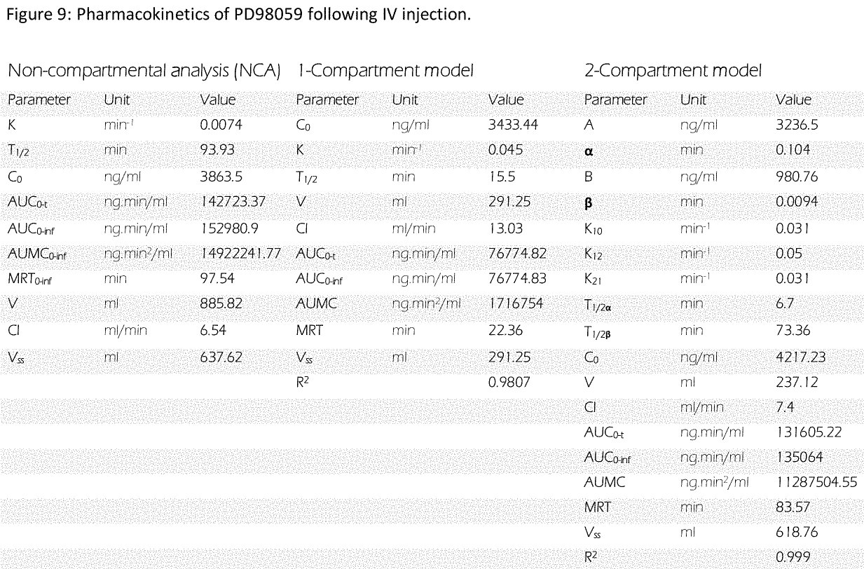

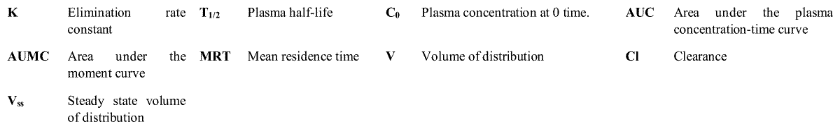

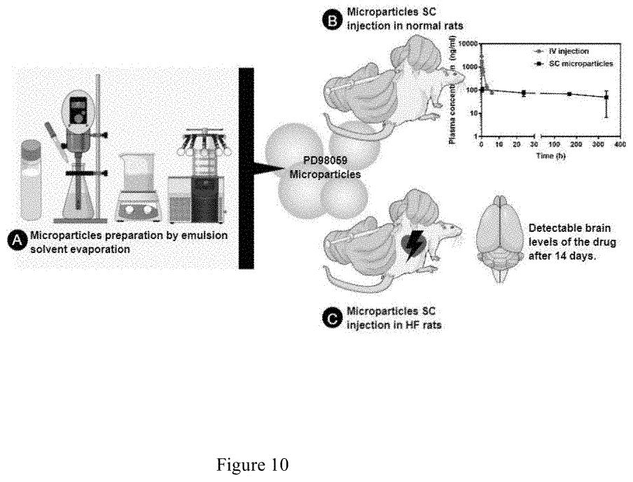

The PD98059 HPLC' peak came after 3 min, while the retention time of the IS (7-hydroxyflavone) was 3.6 min. The pharmacokinetics of PD98059 were studied after the data were fit to 1-compartment or 2-compartments models using PK Solver add-in. It can be clearly seen that PD98059 pharmacokinetics follow the 2-compartment model (FIG. 6a), which was confirmed by the R² value (0.999 and 0.981 for 2- and 1-compartment models, respectively). Pharmacokinetics parameters are displayed in FIG. 9. Distribution and elimination half-lives of the drug were approximately 7 and 73 minutes, respectively. After 1 and 3 hours, levels of PD98059 in major organs were also measured (n=3) in order to gain information on tissue distribution and organ drug levels decline. It can be seen that the drug levels in the liver and kidney were comparatively higher than those in the brain and heart, and yet a significant portion of the drug crossed the blood brain barrier. Nevertheless, there was a quick decline in brain PD98059 levels from approximately 410 ng/g to 76 ng/g between 1 hour and 3 hours.

### Pharmacokinetics and Brain Levels of PD98059 Following Subcutaneous Injection PD98059-Loaded Microparticles

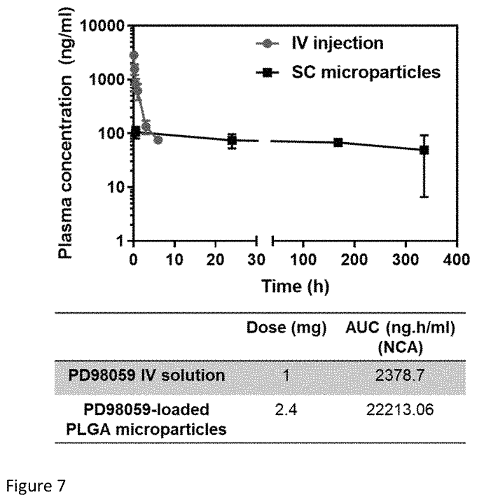

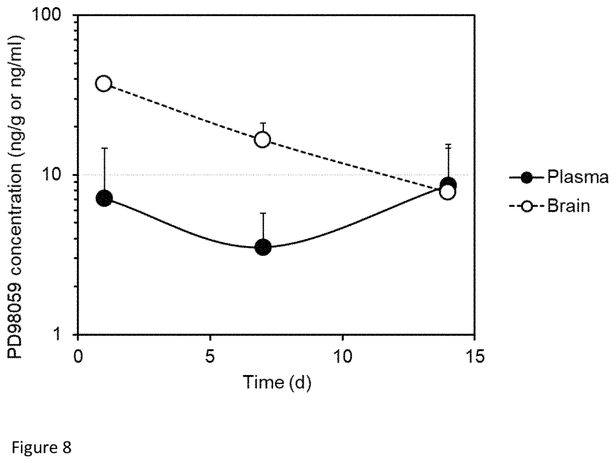

In the normal rats treated subcutaneously with PD98059-loaded microparticles, there was a sustained level of PD98059 in the plasma of rats of 50-100 ng/ml over a period of 2 weeks (FIG. 7). It was found that the AUC₀₋ₜ values following the SC injection of 2.4 mg of PD98059 in PLGA microparticles was 22213.1 ng.h/ml, compared with 2378.7 ng.h/ml following IV injection of 1 mg of PD98059 in solution.

The brain levels of PD98059 in the heart failure rats declined gradually over the course of 2 weeks. The levels in the brain were found to be approximately 37, 16, and 8 ng/g after 1, 7, and 14 days, respectively. The plasma levels were found to be about 7.1, 3.5, and 8.6 ng/ml after 1, 7, and 14 days of injection of the microparticles.

### Discussion

Previous work highlighted the ability of centrally administered PD98059 to lower p-ERK1/2 levels in the PVN of rats with heart failure, with abrogation of the sympathetic excitation that contributes to further deterioration of cardiac function. Such findings pave the way towards development of a new therapeutic modality in drug-based treatments of heart failure and introduces the new concept of using small molecules to target the central nervous system mechanisms driving sympathetic excitation in heart failure. Researchers in the cancer chemotherapy field have also successfully used inhibitors of the MEK-ERK pathway to potentiate existing cancer treatments. In both cases, the short half-life and reversibility of ERK1/2 inhibitory activity of most of these inhibitors have been major problems that have hindered their progression to the clinical applications. These two problems have necessitated either the repeated administration of these agents at high dosing frequencies, or the development of MEK-ERK inhibitors with long half-lives.

As discussed herein, a PLGA microparticle formulation was formulated that was capable of slow release of PD98059, which is a specific MEK inhibitor with a versatile application spectrum and high clinical potential in many conditions, including cancer and heart failure. Although there was significant variability in plasma and brain levels of PD98059 in this small number of animals, the findings suggest that PD98059 microparticles have a four-fold higher bioavailability, based on the dose-normalized AUC, compared to IV injection of soluble PD98059 (FIG. 7). The rapid decline in drug plasma levels following IV injection (FIG. 6) can be explained based on its relatively high volume of distribution, with a t_{1/2α} as short as 6,7 min, and the quite short elimination half-life of 73 min (FIG. 9). Those two factors were compensated by the continuous slow release of PD98059 from the microparticles at the SC injection site, which guaranteed a steady plasma level in healthy rats (FIG. 7). The pharmacokinetics of PD98059 were best fit to a 2-compartment model (FIG. 9). To maintain sustained levels in the brain of a drug that has a short half-life, it is crucial to maintain a continuous supply. PLGA.-based microparticles are commonly used to provide sustained drug release in the body following intramuscular (IM) or SC injection. PLGA is a bulk-eroding polyester from which the drug release usually follows a biphasic pattern, comprising an initial diffusion-based release which extends for a few days to a few weeks (depending on the molecular weight of the polymer), and a subsequent constant release phase explained by erosion of the matrix combined with some contribution from the declining diffusion.

The microparticles had an encapsulation efficiency of 43% and an average size of 16.7 μm in diameter. The drug release was monitored over 4 weeks, even though the plasma levels were detected for only two weeks. Plasma drug levels after 3 and 4 weeks could not be detected by HPLC. The in vitro release study showed that 55% of the drug was released within the first two weeks, compared to a further 19% over the next two weeks (between weeks 2 and 4). This marked reduction of drug release rate after the second week may explain the absence of measurable drug levels in the plasma during this time, taking into account the short half-life of the drug.

Thus, subcutaneous administration of a sustained-release microparticle preparation might modulate the excess ERK1/2 activity in cardiovascular regions of the brain that drive sympathetic excitation in a rat model of heart failure. Heart failure alters the pharmacokinetics of many drugs, mainly BCS class II and IV drugs (i.e. those with poor solubility and good permeation and those with poor solubility and poor permeability, respectively), and absorption of these drugs following oral administration was described as erratic, delayed, and poor. Shammas and Dickstein reported that the reduced blood flow to the muscles in congestive heart failure adversely affects the absorption of poorly water soluble drugs and thus their intramuscular administration should be avoided. More importantly, in a study on 46 patients, Ariza-Andraca reported that the rate and extent of absorption of subcutaneously injected insulin was significantly lowered in diabetic patients with generalized edema. The insulin amount absorbed after 6 hours was three to four times less in diabetic patients with edema compared to patients without edema. The authors related this sharp decline in rate and extent to subcutaneous edema.

In the present study, plasma levels in rats with established heart failure injected with PD98059-loaded microparticles were lower and more variable than those in healthy rats (FIG. 9), which may be a result of decreased subcutaneous absorption in heart failure rats, the use of different rats at different time points, or both. Nevertheless, plasma levels, and of more significance brain levels, were still detectable for up to two weeks. Previously, HF rats were treated with PD98059 solution via ICV for 1 h and 4 weeks and obtained therapeutic effects in both cases. For the short-term experiment (1 h), the dose was 40 μl/h of 20 μM solution of PD98059 for 1 hour. This is equivalent to approximately 214 ng/h. in the long-term experiment (4 weeks), the dose was 0.25 ul/h of 0.6 mM solution of PD98059 for 4 weeks. This is equivalent to approximately 40 ng/h. The present data show that the brain levels are approximately 40 ng/g of brain tissue after 24 h, which makes the amount delivered to the brain (average weight 15-2 g) about 60-80 ng. The brain levels declined later, reaching about 8 ng/g after 2 weeks. Thus, higher doses of PD98059 may allow for maintenance of constant brain levels for extended periods in HF.

Current heart failure therapy has little impact on central nervous system mechanisms contributing to sympathetic excitation. The prospect of targeting a central pathway regulating sympathetic outflow in heart failure with a systemically administered long-acting drug preparation has clear translational potential.

Despite being widely used in in vitro testing, very few in vivo applications, and no clinical trials have been reported for PD98059. Sufficient pharmacokinetics information is difficult to find/unavailable in the literature. This report describe the pharmacokinetics of this drug, and also gives a hint about its biodistribution in major organs. Also, it is worth mentioning that this is one of the very few reports to shed some light on the effect of heart failure on subcutaneous absorption of drug molecules from a slow release microparticles. In one embodiment, the microparticle preparation provides for high steady-state plasma and brain levels of a MEK1/2 inhibitor such as PD98059 for prolonged periods (1-2 months) thereby allowing for long-term inhibition of PVN pERK1/2 levels and reducing sympathetic activation in heart failure.

### Conclusion

PD98059, a potent but reversible MEK inhibitor, has a short elimination half-life, barely above 1 hour, in rats. When combined with a reversible MEK inhibitory activity, a continuous supply of the drug needs to be provided in order to achieve long-term therapeutic goals. PD98059-loaded. PLGA microparticles were successfully prepared and characterized using emulsion solvent evaporation technique. The prepared microparticles produced steady plasma levels of PD98059 in rats following SC injection, Detectable levels of PD98059 in the brain were also present for up to two weeks in rats with heart failure, encouraging the further development of this formulation for long-term inhibition of p-ERK1/2 in brain regions like PVN that contributes to the increased sympathetic nerve activity in heart failure and for use in cancers in which ERK1/2 activity may contribute to progression.

### SUMMARY

The pharmacokinetics of PD98059 that was dissolved in a U.S. FDA-approved vehicle (10% v/v Tween 80 in sterile phosphate buffer saline (PBS), pH 7.4) and injected intravenously (W) in normal rats was determined. A formulation was employed to overcome its short plasma half-life and take advantage of its ability to cross the blood-brain barrier. In one embodiment, a poly lactide-co-glycolide (PLGA) microparticle formulation of a EK1/2 inhibitor is employed to prevent, inhibit or treat heart failure. In one embodiment, the inhibitor is in a sustained release formulation, e.g., a PLGA. formulation, that is biocompatible and biodegradable, such as a polyester, that provides sustained release of small molecules and macromolecules alike. Slow drug release from the bulk-eroding polymer matrix not only provides sustained plasma concentrations at therapeutic levels, but also prevents sharp peaks and troughs in plasma levels that can result from multiple administrations and may result in toxicity or sub-therapeutic levels.

The PLGA formulation provides a sustained steady plasma drug level that is able to facilitate passage of PD98059 into the brain as shown in normal rats and rats with heart failure, whose compromised circulation might adversely affect subcutaneous absorption, over a two-week interval following a single subcutaneous injection.

### EXAMPLE 3

Research over the past fifteen years revealed that effective control of SNA in HF rats can be achieved via prolonged inhibition of p-ERK1/2 levels in the PVN. The highly selective MFK1/2 inhibitor PD98059 was selected to inhibit p-ERK1/2 levels in the PVN and was found to cross the blood brain barrier. However, its short half-life and reversible mode of action are obstacles that need to be tackled in order to achieve long-term activity, Previously, long term ICV infusion of the drug successfully decreased PVN p-ERK1/2, and subsequently plasma NE levels in HF rats, however, the highly invasive nature of this procedure diminished its clinical feasibility. As disclosed herein, an alternative way to achieve long-term PVN p-ERK1/2 inhibition is via subcutaneous (SC) injection of sustained-release poly lactide-co-glycolide (PLGA) microparticles loaded with PD98059 in HF rats. Two weeks post-injection, p-ERK1/2 levels were significantly decreased (p<0.05) compared to vehicle-treated HF rats. Circulating NE levels also decreased significantly. The formulation did not exhibit any noticeable toxicity in HT rats compared to untreated HF rats. Thus, the efficacy, apparent safety, and low frequency of administration of this formulation offers a novel approach to the long-term treatment of the central manifestations of HF, as a mono- or adjuvant therapy in combination with other pharmacological agents that act peripherally.

### Introduction

Systolic heart failure (HF) is characterized by exaggerated sympathetic nerve activity (SNA), which is one culprit behind further heart performance deterioration. The hypothalamic paraventricular nucleus (PVN) is a region in the forebrain rich in presympathetic neurons that regulate most neurohumoral responses related to sympathetic excitation. In HF, the neurochemical signals that control sympathetic activity in the PVN are massively deranged, with a subsequent increased RAS activity, endoplasmic reticulum (ER) stress, and elevated levels of proinflammatory cytokines. Central interventions that interfere with these neurochemical abnormalities consistently inhibit SNA and improve the peripheral manifestations of HF.

The mitogen-activated protein kinase (MAPK) cascade is an evolutionarily conserved protein kinase pathway crucial for several biological functions inside the mammalian cells, including cell survival, proliferation, and apoptosis, among others. In Ras-Raf-MEK-ERK pathway, which is one of the most widely studied MAPK pathways, the activation of the GDP/GTP binding protein Ras is followed by the activation of Raf (also called MAPK kinase kinase, or MAPKKK). Raf activation activates MEK (MAPK-ERK kinase), which is followed by ERK1/2 (extracellular signal-regulated kinase, also known as p42/44) activation.

Research over the past fifteen years revealed that the Ras-Raf-MEK-ERK pathway in the PVN plays a fundamental role in the sympathetic excitation that accompanies, and ultimately aggravates, heart failure in rats. In heart failure, it was found that sympathetic excitation that originates in the PVN by the action of the upregulated excitatory agonists takes place following activation of the downstream kinase ERK1/2 in the PVN. As a result, higher levels of phosphorylated ERK1/2 (pERK1/2) were found in the PVN of HF rats, along with PVN neuronal excitation PVN neuronal excitation and sympathetic nerve activity were inhibited by a short-term 1-hour intracerebroventricular (ICV) infusion of PD98059, a specific MEK1/2 inhibitor, in heart failure rats. Long-term ICV infusion of PD98059 (for 4 weeks) normalized plasma levels of norepinephrine in heart failure rats, which is an indication of decreased sympathetic nerve activity.

PD98059 was chosen in these studies because it was found to cross the blood brain barrier (BBB) to reach the PVN (sequestered behind the BBB) at therapeutic levels. The short half life of PD98059 and its reversible MEK1/2 inhibition activity are the two major hurdles that impede the progress of the application of PD98059 in the clinic. The elimination half-life of PD98059 following IV injection of a solution of the drug in rats was around 70 min. Earlier, Dudley et al. reported that the in vitro activity of MEK was fully and instantaneously restored once PD98059 was removed from the medium.

### Materials

PD98059 was purchased from Selleck Chemicals (Houston, Tex.). Poly (lactide-co-glycolide) (PLGA, Resomer RG 503) was purchased from Evonik (Parsippany, N.J.). Poly vinyl alcohol (Mowiol 8-88, MW 67,000) was purchased from Sigma Aldrich (St Louis, Mo.). Tween 80 was purchased from Fisher Chemicals (Waltham, Mass.). All other chemicals were of analytical grade and used without further purification.

Microparticle Preparation

Microparticles were prepared using single emulsion solvent evaporation technique. Briefly, 2.75 mg of PD98059 were dissolved in 300 ul of dichloromethane (DCM). Then, 200 mg of PLGA were also dissolved in 1.2 ml of DCM, and the two solutions were combined in a single vial. This organic solution (oil phase) was added to an aqueous phase composed of 30 ml of 1% w/v polyvinyl alcohol (PVA) in 150 ml beaker. The mixture was homogenized (Ultra-turrax T25 basic. Ika Works Inc., Wilmington, N.C.) at 17500 rpm for 30 seconds. Solvent evaporation was achieved following stirring of the emulsion at room temperature at 600 rpm for 90 minutes under a fume hood. The microparticles were collected by centrifugation at 1000×g for 5 min (Eppendorf Centrifuge 5864 R, Eppendorf, Hauppauge, N.Y.) and the supernatant was rejected. The microparticles were washed twice in 30 ml Nano-Pure water (Barnstead Thermolyne Nanopure water. Thermo Fisher, Waltham, Mass.) followed by centrifugation. The microparticles were then resuspended in 5 ml of Nano-Pure water, frozen at −80° C., and lyophilized overnight (Labconco Free zone 4.5, Labconco, Kansas City, Mo.).

### Microparticle Characterization

### Microparticle Morphology

Scanning electron microscopy (SEM) was used to examine the microparticles morphology. A thin sheet of the lyophilized microparticles on a carbon double-tape mounted on an aluminum SEM stub was sputter-coated with gold and palladium (Emitech K550 sputter-coater). The SEM images were taken using Hitachi S-4800 scanning electron microscope (Hitachi High Technologies America. Inc., Schaumburg, Ill.). The particle sizes of about 70 particles in SEM images were measured using Image) (NIH, Bethesda, Mass.) and the data were plotted using Microsoft Excel.

### Drug Content Determination

A weighed amount of the microparticles was dissolved in DCM at a concentration of 1 mg/ml. One hundred microliters of this solution were diluted with methanol to 6.5 ml, and finally centrifuged at 12,000×g for 5 min. The supernatant was further diluted with purified water to 10 ml, and the concentration of the resultant solution was measured by HPLC as mentioned below.

Drug content (μg/mg) of the microparticles was calculated using equations 1, as follows:

$$Drug content ( \frac{μg}{mg} ) = \frac{amount of PD98059 ( μg )}{weight of microparticles after lyophilization ( mg )} \qquad Equation 1$$

### In Vitro Drug Release

A weighed amount of the microparticles was suspended in 5 ml of the release medium (Dulbecco's phosphate buffered saline, DPBS, Life Science, Waltham, Mass.) that contains 0.4% w/v Tween 80 at an amount equivalent to 0.065 mg microparticles in 50 ml tube (n=3). The tubes were placed in an orbital shaker (New Brunswick Scientific, Edison, N.J.) operating at 300 rpm and 37° C. At pre-determined time points, the tube was centrifuged (1000×g for 5 min) and the whole volume of the release medium (5 ml) was removed and replaced completely with fresh medium in which the pellet was re-suspended. The concentration of PD98059 was measured in the samples using the HPLC method described below. The standard curve range was 0,125-10 μg/ml, and the r² value was 0.9998.

### Experimental Protocol

Nine Male Sprague-Dawley rats (6-8 weeks) weighing about 275-330 g, obtained from Envigo. Indianapolis, Ind. were used in the experiment and were kept at the University of Iowa animal care facility. They were kept under controlled temperature at around 23±2° C. and were exposed to 12-hours of light and dark cycles. Food was provided to the rats ad libitum. All animal experiments performed were approved by the University of Iowa Institutional Animal Care and Use Committee.

Heart failure in anesthetized rats (ketamine/xylazine) was induced by ligation of the left coronary artery under sterile conditions. Twenty four hours later, heart failure was confirmed by echocardiography in the form of reduced systolic function (with rats showing left ventricular ejection fraction of less than 40%), then the microparticles suspension (PD98059-loaded or blank, n=4-5/group) was injected SC in the rats. The microparticles suspension for SC injection was prepared by suspending an accurately weighed amount of lyophilized PD98059-loaded microparticles (containing 0.4 mg of PD98059/rat), or an equal amount of the blank microparticles, in 1 ml of 1× DPBS and injected SC in the shaved back of each rat.

Two weeks after the microparticles injection, the HF rats were euthanized by decapitation following urethane anesthesia, then their brains were collected, and total ERK1/2 and p-ERK1/2 levels were determined by Western blot analysis in PD98059-loaded microparticles rats (n=5), and blank microparticles rats (n=4). Protein levels of total ERK1/2, and p-ERK1/2, and β-actin were analyzed by Western blot analysis using primary antibodies to p-ERK1/2 and β-actin (Cell Signaling Technology, Danvers, Mass.). The bands densities were quantified using Image Lab analysis software (Bio-Rad, Hercules, Calif.).

Plasma norepinephrine (NE) levels in HF rats (n=5 for PD98059-loaded microparticles rats and n=4 for blank microparticles rats) were measured by an ELISA kit (Rocky Mountain Diagnostics, Colorado Springs, Colo.) according to the manufacturer's instructions.

### HPLC

An Agilent Infinity 1100 HPLC (Santa Clara, Calif.) was used for to analyze PD98059 content and release of the microparticles. The HPLC consisted of a quaternary pump (Agilent Technologies), diode array detector (DAD, Agilent Technologies) and auto-injector (Agilent Technologies). A Waters Symmetry Reversed phase C-18 (RP-C18) column was used for PD98059 assay (5 μm, 4.6 mm×150 mm, Milford, Mass.). The mobile phase composition was methanol: water 70:30 with 0.1% v/v trifluoroacetic acid (TFA). The flow rate used was 1 ml/min at room temperature, and the wavelength at which PD98059 was detected at 275 nm.

### Statistical Analysis

All data presented are means±SD, Statistical significance was analyzed by one-way ANOVA followed by Tukey's post-hoc test or Student T-test. Data were considered significant if p value is <0.5.

### Results

As disclosed herein, a single injection of a sustained-release dosage form of PD98059, i.e., PD98059-loaded biodegradable microparticles was able to significantly decrease the elevated levels of p-ERK1/2 in the PVN and NE in plasma of HF rats for up to 2 weeks. This long,-term efficacy was only achievable using continuous long-term delivery of PD98059 by means of a highly invasive non-clinically practical ICV infusion for 4 weeks.

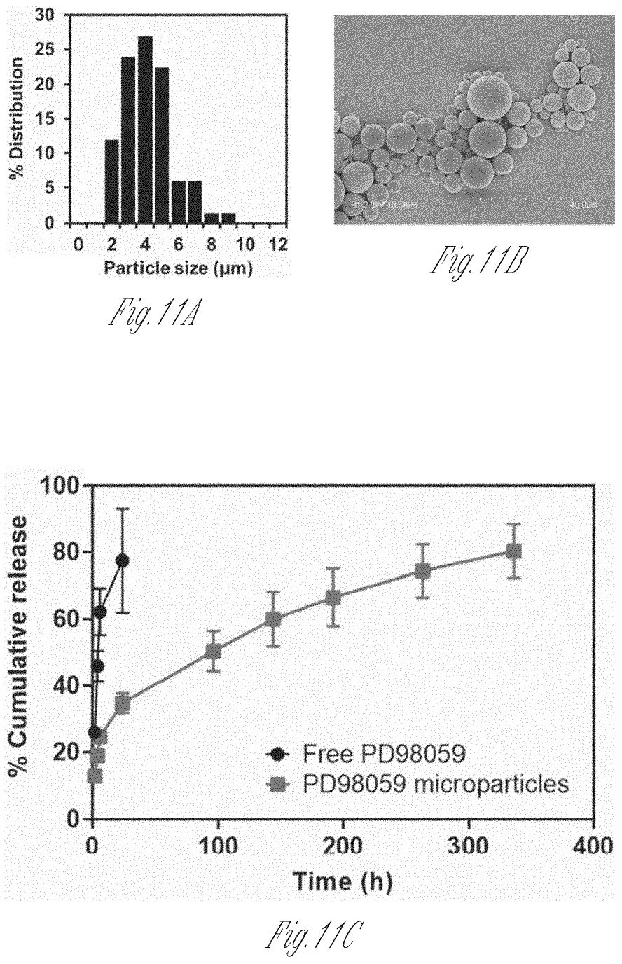

Poly lactide-co-glycolide (PLGA, Resomer RG502, lactide: glycolide ratio of 50:50, molecular weight 24-38 kDa, Evonik, Birmingham, Ala.), a biodegradable polyester that degrades in vivo into biocompatible by-products, was used to prepare the microparticles. The microparticles were spherical and had smooth surface, as can be seen in the scanning electron microscopy image (FIG. 11a). The average particle size (FIG. 11b) and drug loading of these particles were found to be around 4 μm and 13.22±1.24 μg drug per mg microparticles, respectively. Detailed experimental procedure for microparticles preparation and characterization can be found in the supplementary section. The microparticles exhibited slow drug release, as approximately 60% of the loaded drug was released in the first week, and about 80% were released after two weeks. Meanwhile, it took only 24 hours for 80% of the unencapsulated drug to dissolve in the release medium. This shows the ability of the microparticles to control the drug release in vitro.

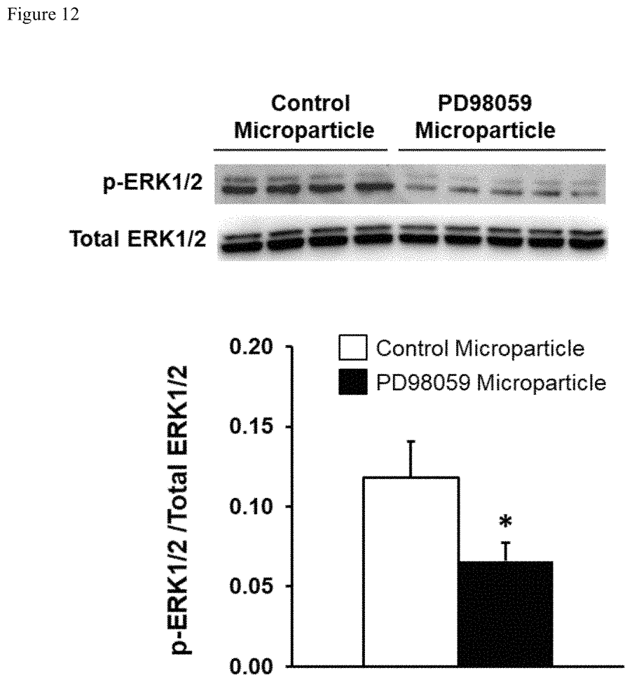

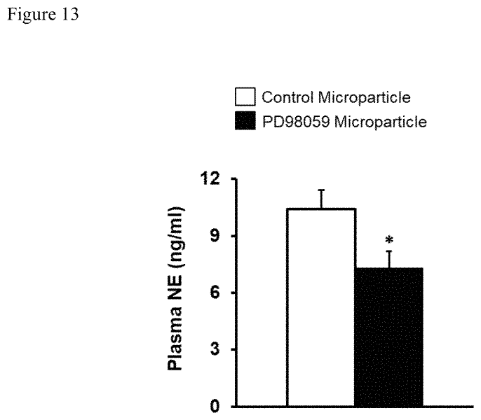

Heart failure was induced in rats by ligation of the left coronary artery. Heart failure was confirmed by echocardiography, then microparticles were injected SC at a dose of 400 μg per rat in I ml of Dulbecco's phosphate buffer saline (DPBS, pH 7.4, Thermo Fisher, Waltham, Mass.). Two weeks after microparticle injection, p-ERK1/2 levels in the PVN (normalized to total ERK1/2) in rats treated with PD98059-loaded microparticles were found to be about half of those in rats treated with blank microparticles (p<0.05, FIG. 12).

As a result of suppression of the SNA originating from the PVN following a long-term inhibition of p-ERK1/2 in this region, we sought to determine whether this is reflected systemically. Plasma norepinephrine (NE) level is used as a general indicator of sympathetic activity. Circulating NE level decreased significantly after two weeks of SC injection of PD98059-loaded microparticles in BF rats, compared to HF rats injected with empty PLGA microparticles (FIG. 13). Previously we showed that a significant reduction of NE plasma levels was achieved following the chronic treatment of HF rats with ICV infusion of the MEK1/2 inhibitor PD98059, and to a lesser extent the P38 MAPK inhibitor SB203580 for 4 weeks. This was not achieved when the c-Jun N-terminal kinase inhibitor SP600125 was given to HF rats in the same manner.

No evidence of toxicity or adverse events was found following the SC injection of these microparticles in HF rats. Echocardiographic investigation did not reveal any negative effect on the left ventricular ejection fraction (LVEF). There was also no kidney toxicity, as outlined by detecting cystatin C levels and kidney injury molecule 1 (KIEM-1) mRNA levels, and no liver toxicity as found by detecting aspartate aminotransferase (AST), alanine aminotransferase (ALT), and bilinibin serum levels compared to HF rats with no treatment. The body weight was not altered, and no behavioral changes were noticed for two weeks after the injection

Congestive heart failure is a major cause of peripheral edema, which, in addition to poor perfusion, are expected to adversely affect extravascular drug absorption. The present data showed that the microparticles were able to provide therapeutic levels of PD98059 to the brain in spite of poor perfusion that may hinder the drug's absorption.

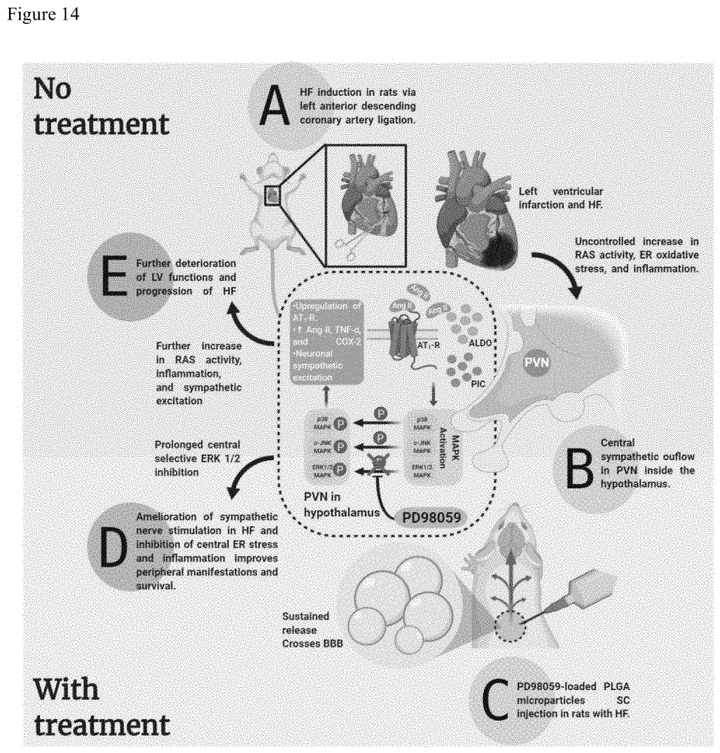

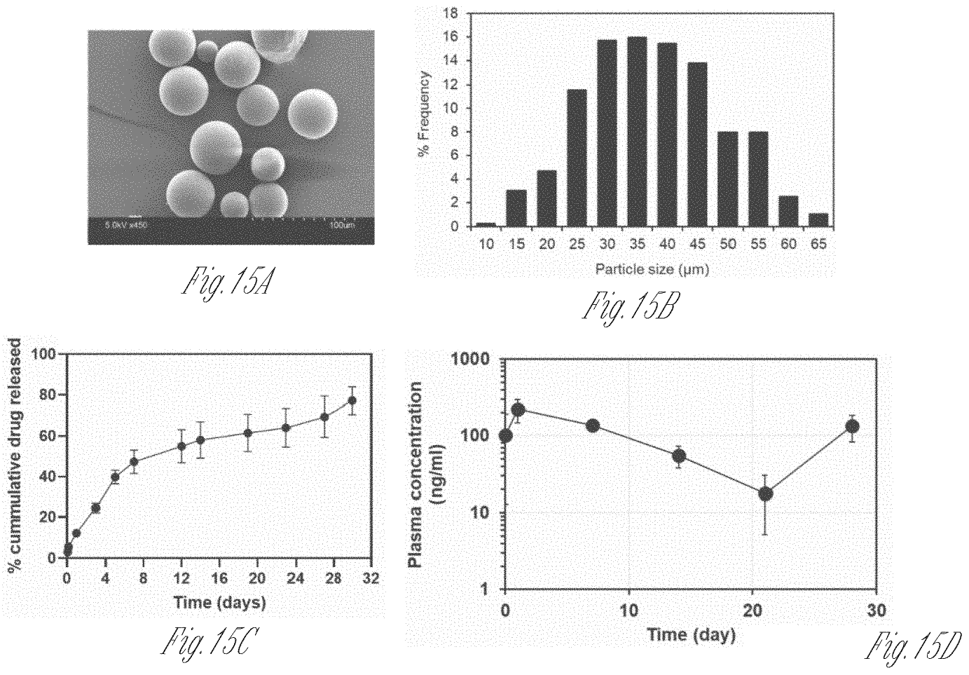

In summary, a sustained release formulation that efficiently curbed the sympathetic excitation in HF rats for up to two weeks was identified. This was achieved through prolonged inhibition of p-ERK1/2 in the PVN, the mastermind that controls sympathetic neurochemical signals that are massively deranged in HF. A MEK1/2 inhibitor that was found to cross the blood brain barrier, PD98059, was slowly released over 2 weeks to keep therapeutic levels in the PVN, sufficient to suppress p-ERK1/2 levels (FIG. 14) This was only achieved using a highly invasive, clinically non-feasible approach. Two problems associated with PD98059 were successfully overcome using this approach; its short half-life and its reversible activity. Long-term therapy that provides prolonged inhibition of p-ERK1/2 in the PVN for up to 3 months may provide an enhanced benefit. This approach may be employed as a single therapy or an adjuvant therapy with other peripheral treatments (e.g. angiotensin converting enzyme (ACE) inhibitors, or β-blockers) to control increased sympathetic activity in HF.

### EXAMPLE 4

A formulation having higher drug loading and higher plasma levels, e.g., in male rats, is described below.

Preparation of Large PD98059-PLGA Microparticles with High Loading

In a 150 ml beaker, add 30 ml of 1% PVA (Mowiol 88-8, MWt 68,000, Sigma) then dissolve 100 g or 100 mg of PLGA (Resomer RG 503, Evonik) and 10 mg of PD98059 (Selleck Chem) in 1.5 ml of DCM. Place the beaker on a jack below the paddle of an overhead stirrer (Talboys Model 101 overhead mixer, USA). Raise the jack so that the paddle is just above the bottom of the beaker. Start overhead stirrer at speed 3.4. Transfer the DCM solution into Pasteur piper, and submerge the Pasteur pipet under the surface of the aqueous phase and start adding the organic phase into the aqueous phase slowly. Avoid touching the metal paddle or shaft or the beaker wall. After addition, keep the stirrer on for 4 minutes (including addition time). Keep a magnetic stirrer (Corning, USA) just next to the overhead stirrer. Once homogenization finishes (after 4 min), transfer the beaker to the magnetic stirrer (at speed 6) after a 1 inch magnetic bar is added to the beaker, and keep it for 2 hours to evaporate DCM. Transfer the microparticles into 50 ml Falcon tubes. Wash the microparticles by centrifugation at 1000×g for 5 min. repeat the washing step twice using 45 ml of Nanopure water during each wash. After the last wash, remove the water completely, then freeze the microparticles at −80° C. for at least 2 h, then lyophilize overnight (Labconco, Freezone 4.5, USA). The morphology of the microparticles was then studied using SEM as mentioned previously. The particle size analysis was performed using ImageJ by counting 370 microparticles from SEM images.

### Drug Content Measurement

Dissolve microparticles at a concentration of 1 mg/ml in DCM. Transfer 100 μl of this solution into a 20 ml scintillation vial, then add 6.4 ml of methanol and vortex and sonicate to dissolve. Add 3.5 ml of Nanopure water, vortex mix, and centrifuge (16,000 ×g, 5 min). Inject the supernatant directly in the HPLC using the method mentioned above. Encapsulation efficiency was calculated using the same equation mentioned above.

### Drug Release

An accurately weighed amount of microparticles (e.g., equivalent to 64 μg of PD98059) was suspended in 15 ml tube containing 5 ml of the release medium (phosphate buffer saline pH 7.4 in Nanopure water with 0.4% v/v Tween 80). Tubes were shaken at 37° C. at 300 rpm in an orbital shaker. At predetermined time points, the tubes were centrifuged at 1000×g for 10 min and 1 ml samples were withdrawn from each tube. The whole media was then discarded and replaced with fresh media and the microparticles were redispersed. Samples were analyzed directly by HPLC as mentioned above.

### Pharmacokinetic Study

Sprague:-Dawley rats (6-8 weeks, 275-300 g, Harlan labs. Indianapolis, Ind.) were injected with the large PD98059-loaded microparticles. Blood samples were collected from the rats at pre-determined time points, and the drug was extracted from the plasma and analyzed by LC-MS using the same method mentioned above.

### Results

The microparticles were spherical in shape (FIG. 15A), with a size range of 35-40 μm (FIG. 15B). Drug release took place over 1 month, releasing about 80% of the drug loaded within this period (FIG. 15C).

A pharmacokinetic study revealed that the microparticles kept a sustained plasma level in rats for about 4 weeks, Plasma levels reached about 222 ng/ml after 24 h, then started to decline afterwards until they reached about 18 ng/ml, after which the levels increased again during the fourth week up to approximately 134 ng/ml (FIG. 15D). This correlates well with the in vitro release, where the drug release rate increased between the third and fourth weeks after a period of slow release.

### REFERENCES

Andhariya J V, Sheri J, Choi S, Wang Y, Zou Y, Burgess D J. Development of in vitro-in vivo correlation of parenteral naltrexone loaded polymeric microspheres. Journal of controlled release: official journal of the Controlled Release Society. 2017; 255:27-35. doi:10.1016/j.jconrel. 2017.03.396.

Ansary R H, Rahman M M, Awang M B, Katas H, Hadi H, Doolaanea A A. Preparation, characterization, and in vitro release studies of insulin-loaded double-walled poly(lactide-co-glycolide) microspheres. Drug delivery and translational research. 2016; 6(3):308-18. doi:10.1007/s13346-016-0278-y.

Ariza-Andraca C R, Altamirano-Bustamante E, Frati-Munari A C, Altamirano-Bustamante P, Graef-Sanchez A. Delayed insulin absorption due to subcutaneous edema. Archivos de investigacion medica. 1991; 22(2):229-33.

Awasthi N, Monahan S, Stefaniak A, Schwarz M A, Schwarz R E. Inhibition of the MEK/ERK pathway augments nab-paclitaxel-based chemotherapy effects in preclinical models of pancreatic cancer. Oncotarget. 2018 ;9(4):5274-86. doi:10.18632/oncotarget.23684.

Basu S, Harfouche R, Soni S, Chimote G, Mashelkar R A, Sengupta S. Nanoparticle-mediated targeting of MAPK signaling predisposes tumor to chemotherapy. Proceedings of the National Academy of Sciences of the United States of America. 2009; 106(19)7957-61. doi:10,1073/pnas.0902857106.

Blaukat A, Barac A, Cross M J, Offermanns S, Dikic I. G protein-coupled receptor-mediated mitogen-activated protein kinase activation through cooperation of Galpha(q) and Galpha(i) signals. Molecular and cellular biology. 2000; 20(18):6837-48. doi:10.1128/mcb.20.18.6837-6848.2000.

Broman K K, Dossett L A, Sun J, Eroglu Z, Zager J S. Update on BRAF and MEK inhibition for treatment of melanoma in metastatic, unresectable, and adjuvant settings. Expert opinion on drug safety. 2019; 18(5):381-92.
doi:10.1080/14740338.2019.1607289.

Chang F, Steelman L S, Lee J T, Shelton J G, Navolanic P M, Blalock W L et al. Signal transduction mediated by the Ras/Raf/MEK/ERK pathway from cytokine receptors to transcription factors: potential targeting for therapeutic intervention. Leukemia. 2003, 17(7):1263-93. doi:10.1038/sj.leu.2402945.

Chang L, Karin M. Mammalian MAP kinase signalling cascades. Nature, 2001; 410(6824)37-40. doi:10.1038/35065000.

Cheng Y, Tian H. Current Development Status of MEK Inhibitors. Molecules. 2017; 22(10). doi : 10.3390/molecules22101551.

Cilurzo F, Selmin F, Minghetti P, Montanari L. Design of methylprednisolone biodegradable microspheres intended for intra-articular administration. AAPS PharmSciTech. 2008, 9(4):1136-42. doi:10.1208/s12249-008-9158-1.

Costigan D C, Doug F. The extended spectrum of RAS-MAPK pathway mutations in colorectal cancer. Genes, chromosomes &. cancer. 2019, doi:10.1002/g,cc.22813.

Cseh B, Donna E, Baccarini M. “RAF” neighborhood: protein-protein interaction in the Raf/Mek/Erk pathway. FEBS letters. 2014, 588(15):2398-406. doi: 10.1016/j.febslet. 2014.06.025.

Dudley D T, Pang L, Decker S J, Bridges A J, Saltiel A R. A synthetic inhibitor of the mitogen-activated protein kinase cascade. Proceedings of the National Academy of Sciences of the United States of America, 1995; 92(17):7686-9. doi:10.1073/pnas.92. 17.7686.

Felder R B, Yu Y, Zhang Z H, Wei S G, Pharmacological treatment for heart failure: a view from the brain. Clinical pharmacology and therapeutics. 2009; 86(2):216-20. doi:10.1038/clpt.2009.117.

Geary S M, Hu Q. Joshi V B, Bowden N B, Salem A K. Diaminosulfide based polymer microparticles as cancer vaccine delivery systems. Journal of controlled release: official journal of the Controlled Release Society. 2015; 220(Pt B):682-90. doi:10.1016/j.jconrel2015.09.002.

Gilmartin A G, Bleam M R, Groy A. Moss K G, Minthorn E A, Kulkarni S G et al. GSK1120212 (JTP-74057) is an inhibitor of MEK activity and activation with favorable pharmacokinetic properties for sustained in vivo pathway inhibition. Clinical cancer research : an official journal of the American Association for Cancer Research. 2011, 17(5):989-1000. doi:10.1158/1078-0432.CCR-10-2200.

Hong L, Krishnamachari Y, Seabold D, Joshi V, Schneider G, Salem A K. Intracellular release of 17-beta. estradiol from cationic polyamidoamine dendrimer surface-modified poly (lactic-co-glycolic acid) microparticles improves osteogenic differentiation of human mesenchymal stromal cells. Tissue engineering Part C, Methods. 2011; 17(3):319-25. doi:10.1089/ten.TEC.2010.0388.

Intra J, Salem A K. Fabrication, characterization and in vitro evaluation of poly(D,L-lactide-co-glycolide) microparticles loaded with polyamidoamine-plasmid DNA dendriplexes for applications in nonviral gene delivery. Journal of pharmaceutical sciences. 2010; 99(1):368-84. doi:10.1002/jps,21840.

Kang Y M, Zhang Z H, Xue B, Weiss R M, Felder R B. inhibition of brain proinflammatory cytokine synthesis reduces hypothalamic excitation in rats with ischemia-induced heart failure. American journal of physiology Heart and circulatory physiology. 2008; 295(1):H227-36. doi:10.1152/ajpheart.01157.2007.

Khaled K A, Sarhan H A, Ibrahim M A, Ali A H, Naguib Y W, Prednisolone-loaded PLGA microspheres in vitro characterization and in vivo application in adjuvant-induced arthritis in mice. AAPS PharmSciTech. 2010; 11(4859-69. doi:10.1208/s12249-010-9445-5.

Lew B, Kim I Y, Choi H, Kim K K, Sustained exenatide delivery via intracapsular microspheres for improved survival and function of microencapsulated porcine islets. Drug delivery and translational research, 2018; 8(3):857-62. doi:10.1007/s13346-018-0484-x.

Li L, Zhao G D, Shi Z, Qi L L, Zhou L Y, Fu Z X. The Ras/Raf/MEK/ERK signaling pathway and its role in the occurrence and development of HCC. Oncology letters. 2016; 12(5):3045-50. doi:10.38921ol.2016.5110.

Li S, Balmain A, Counter C M. A model for RAS mutation patterns in cancers: finding the sweet spot. Nature reviews Cancer. 2018; 18(12):767-77. doi:10.1038/s41568-018-0076-6.

Li S, Liu S, Deng J. Akbay E A, Hai J, Ambrogio C et al. Assessing Therapeutic Efficacy of MEK Inhibition in a KRAS(G12C)-Driven Mouse Model of Lung Cancer. Clinical cancer research: an official journal of the American Association for Cancer Research. 2018; 24(19):4854-64. doi:10.1158/1078-0432.CCR-17-3438.

Miao L, Tian H. Development of ERK1/2 inhibitors as a therapeutic strategy for tumour with MAPK upstream target mutations. Journal of drug targeting. 2019:1-12. doi:10.1080/1061186X.2019.1648477.

Naguib Y W, Lansakara P D, Lashinger L M, Rodriguez B L, Valdes S, Niu M et al, Synthesis, Characterization, and In Vitro and In Vivo Evaluations of 4-(N)-Docosahexaenoyl 2′,2′-Difluorodeoxycytidine with Potent and Broad-Spectrum Antitumor Activity, Neoplasia. 2016; 18W:33-48. doi:10.1016/j.neo.2015.11.012.

Naguib, Y. W., Y. Yu, S. G. Wei, A. Morris, B. E. Givens, A. I. Mekkawy, R. M. Weiss, R. B. Felder and A. K. Salem (2020). “An injectable microparticle formulation provides long-term inhibition of hypothalamic ERK1/2 activity and sympathetic excitation in rats with heart failure.” Mol Pharm 17(9): 3643-3648, DOI: 10.1021/acs.molpharmaceut.0c00501

Naguib, Y. W., B. E. Givens, G. Ho, Y. Yu, S. G. Wei, R. M. Weiss, R. B. Felder and A. K, Salem (2021). “An injectable microparticle formulation for the sustained release of the specific MEK inhibitor PD98059: in vitro evaluation and pharmacokinetics.” Drug Deliv Transl Res 11(1): 182-191. DOI: 10,1007/s13346-020-00758-9

Navas J P, Martinez-Maldonado M. Pathophysiology of edema in congestive heart failure. Heart disease and stroke: a journal for primary care physicians. 1993, 2(4):325-9.

Neuzillet C, Tijeras-Raballand A, de Mestier L, Cros Faivre S, Raymond E. MEK in cancer and cancer therapy. Pharmacology & therapeutics. 2014; 141(2):160-71. doi:10.1016/j.pharmthera.2013.10.001.

Nies A S. Clinical Pharmacokinetics in Congestive Heart Failure. In: Hosenpud J D, Greenberg B H, editors. Congestive Heart Failure: Pathophysiology, Diagnosis, and Comprehensive Approach to Management. New York, N.Y.: Springer New York; 1994. p.323-40.

Ogawa R, Stachnik J M, Echizen H. Clinical pharmacokinetics of drugs in patients with heart failure: an update (part 2, drugs administered orally). Clinical pharmacokinetics. 2014; 53(12):1083-114. doi:10.1007/s40262-014-0189-3.

Parati G, Esler M. The human sympathetic nervous system: its relevance in hypertension and heart failure. European heart journal. 2012; 33(9):1058-66. doi :10.1093/eurheartj/ehs041.

Pearson G, Robinson F, Beers Gibson T, Xu B E, Karandikar M, Berman K et al. Mitogen-activated protein (MAP) kinase pathways: regulation and physiological functions. Endocrine reviews. 2001; 22(2):153-83. doi:10.1210/edrv.22.2.0428.

Savoia P, Fava P, Casoni F, Cremona O. Targeting the ERK Signaling Pathway in Melanoma. International journal of molecular sciences. 2019; 20(6), doi:10.3390/ijms20061483.

Shammas F V, Dickstein K. Clinical pharmacokinetics in heart failure. An updated review. Clinical pharmacokinetics. 1988; 15(2):94-113.
doi:10.2165/00003088-198815020-00002.

Triposkiadis F, Karayannis G, Giamouzis G, Skoularigis J, Louridas G, Butler J. The sympathetic nervous system in heart failure physiology, pathophysiology, and clinical implications. Journal of the American College of Cardiology. 2009; 54(19):1747-62. doi:10.1016/j.jacc.2009.05.015.

Wang A X, Qi X Y. Targeting RAS/RAF/MEK/ERK signaling in metastatic melanoma. IUBMB life. 2013; 65(9):748-58. doi:10,1002/iub.1193.

Wei S G, Yu Y, Weiss R M, Felder R B. Endoplasmic reticulum stress increases brain MAPK. signaling, inflammation and renin-angiotensin system activity and sympathetic nerve activity in heart failure. American journal of physiology Heart and circulatory physiology. 2016; 311(4):H871-H80.
doi:10.1152/ajpheart.00362.2016.

Wei S G, Yu Y, Weiss R M, Felder R B. Inhibition of Brain Mitogen-Activated Protein Kinase Signaling Reduces Central Endoplasmic Reticulum Stress and Inflammation and Sympathetic Nerve Activity in Heart Failure Rats. Hypertension. 2016, 67(1):229-36. doi:10.1161/HYPERTENSIONAHA.115.06329.

Wei S G, Yu Y, Zhang Z H, Felder R B. Angiotensin II upregulates hypothalamic ATI receptor expression in rats via the mitogen-activated protein kinase pathway. American journal of physiology Heart and circulatory physiology. 2009; 296(5):H1425-33. doi:10.11.152/ajpheart.00942.2008.

Wei S G, Yu Y, Zhang Z H, Weiss R M, Felder R B. Angiotensin II-triggered p44/42 mitogen-activated protein kinase mediates sympathetic excitation in heart failure rats. Hypertension. 2008; 52(2):342-50.
doi:10,1161/HYPERTENSIONAHA.108.110445.

Wei S G, Yu Y, Zhang Z H, Weiss R M, Felder R B. Mitogen-activated protein kinases mediate upregulation of hypothalamic angiotensin II type 1 receptors in heart failure rats. Hypertension. 2008; 52(4):679-86.
doi:10.1161/HYPERTENSIONAHA.108.1.13639.

Wei S G, Zhang Z H, Yu Y, Weiss R M, Felder R B. Central actions of the chemokine stromal cell-derived factor 1 contribute to neurohumoral excitation in heart failure rats. Hypertension. 2012; 59(5):991-8.
doi:10.1161/HYPERTENSIONAHA.111.188086.

Wu P K, Park J I. MEK1/2 Inhibitors: Molecular Activity and Resistance Mechanisms. Seminars in oncology. 2015; 42(6):849-62.
doi :10.1053/j.seminoncol.2015.09.023

Yao J, Chian C, Shu T, Zhang X, Zhao Z, Liang Y. Combination treatment of PD98059 and DAPT in gastric cancer through induction of apoptosis and downregulation of WNT/beta-catenin, Cancer biology & therapy. 2013; 14(9):833-9. doi:10.4161/cbt.25332.

Yu Y, Wei S G, Zhang Z H, Weiss R M, Felder R B. ERK1/2 MAPK signaling in hypothalamic paraventricular nucleus contributes to sympathetic excitation in rats with heart failure after myocardial infarction. American journal of physiology Heart and circulatory physiology. 2016; 310(6):H732-9. doi:10.1152/ajpheart.00703.2015.

Yu Y, Xue B J, Zhang Z H, Wei S G, Beltz T G, Guo F et al. Early interference with p44/42 mitogen-activated protein kinase signaling in hypothalamic paraventricular nucleus attenuates angiotensin II-induced hypertension. Hypertension. 2013; 61(4):842-9. doi:10.1161/HYPERTENSIONAHA.111.00080.

Zhang H, Gao S. Temozolomide/PLGA microparticles and antitumor activity against glioma C6 cancer cells in vitro. International journal of pharmaceutics, 2007; 329(1.-2):122-8. doi:10.1016/j.ijpharm.2006.08,027.

Zhang Y, Huo M, Zhou J, Xie S. PKSolver: An add-in program for pharmacokinetic and pharmacodynamic data analysis in Microsoft Excel. Computer methods and programs in biomedicine. 2010; 99(3):306-14.
doi: 10.1016/j.cmpb.2010.01.007.

Zhang Z H, Yu Y, Wei S G, Felder R B. Aldosterone-induced brain MAPK signaling and sympathetic excitation are angiotensin II type-1 receptor dependent. American journal of physiology Heart and circulatory physiology.
2012; 302(3):H742-51. doi:10.1152/ajpheart.00856.2011.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition comprising microparticles or liposomes comprising one or more MEK1/2 inhibitors.

2. The composition of claim 1 wherein one of the inhibitors is a reversible inhibitor.

3. The composition of claim 1 which provides for sustained release of the one or more MEK1/2 inhibitors.

4. The composition of claim 1 wherein the average diameter of the microparticles is about 1 μm to about 10 μm, about 10 μm to about 50 μm, about 10 μm to about 20 μm, about 25 μm to about 50 μm or about 50 μm to about 200 μm.

5. The composition of claim 1 wherein the microparticles are biocompatible and biodegradable.

6. The composition of claim 1 wherein the microparticles are formed of a polyester.

7. The composition of claim 1 wherein the microparticles are formed of a natural polymer.

8. The composition of claim 1 wherein the microparticles are formed of lactic acid, glycolic acid, or combinations thereof.

9. The composition of claim 1 wherein the one or more MEK1/2 inhibitors comprise PD98059, SL327, pimasertib, cobimetinib, selumetinib, refametinib, trametinib, U0126, Ro 09-2210, CI-1040, PD0325901, RO4987655, RO5126766, binimetinib, TAK733, GDC-0623, G-573, E6201, MFK-162, AZD-8330, TAK-733, GDC-0623, WX-554, HL-085, or AS703988/MSC20150138.

10. The composition of claim 1 wherein the amount of the one or more MEK1/2 inhibitors is effective to prevent or inhibit sympathetic nerve activation.

11. The composition of claim 1 wherein the one or more MEK1/2 inhibitors are released over 1 to 3 weeks or 1 to 4 weeks.

12. A method to prevent, inhibit or treat heart failure in a mammal, comprising administering to the mammal an effective amount of the composition of claim 1.

13. The method of claim 12 wherein the mammal is a human.

14. The method of claim 12 wherein the composition is injected.

14. The method of claim 12 wherein the composition is locally administered.

15. The method of claim 12 wherein the composition is systemically administered.

16. A method to prevent, inhibit or treat sympathetic nerve activation in a mammal, comprising administering to the mammal an effective amount of the composition of claim 1.

17. The method of claim 16 wherein the mammal has cancer.

18. The method of claim 16 wherein the mammal is a human.

19. The method of claim 16 wherein the composition is injected.

20. The method of claim 16 wherein the composition comprises microparticles formed of a polymer having a Mw of about 24,000 to about 38,000.
